SEQUENCE LISTING (33 sequences)

SEQ ID NO: 1 - protein, 184 aa, Homo sapiens
MLQMAGQCSQNEYFDSLLHACIPCQLRCSSNTPPLTCQRYCNASVTNSVKGTNAILWTCLGLSLIISLAVFVLMFLLRKINSEPLKDEFKNTGSGLLGMANIDLEKSRTGDEIILPRGLEYTVEECTCEDCIKSKPKVDSDHCFPLPAMEEGATILVTTKTNDYCKSLPAALSATEIEKSISAR

SEQ ID NO: 2 - protein, 5 aa, Artificial Sequence
  Synthetic polypeptide
GGGGS

SEQ ID NO: 3 - protein, 10 aa, Artificial Sequence
  Synthetic polypeptide
GGGGSGGGGS

SEQ ID NO: 4 - protein, 503 aa, Artificial Sequence
  Synthetic polypeptide
MALPVTALLLPLALLLHAARPDIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSSFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 5 - protein, 503 aa, Artificial Sequence
  Synthetic polypeptide
MALPVTALLLPLALLLHAARPDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 6 - protein, 503 aa, Artificial Sequence
  Synthetic polypeptide
MALPVTALLLPLALLLHAARPDIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTHYSMNWVKQAPGKGLKWMGRINTETGEPLYADDFKGRFAFSLETSASTAYLVINNLKNEDTATFFCSNDYLYSCDYWGQGTTLTVSSFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 7 - protein, 18 aa, Artificial Sequence
  Synthetic polypeptide
GSTSGSGKPGSGEGSTKG

SEQ ID NO: 8 - protein, 507 aa, Artificial Sequence
  Synthetic polypeptide
MLLLVTSLLLCELPHPAFLLIPDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 9 - protein, 506 aa, Artificial Sequence
  Synthetic polypeptide
MALPVTALLLPLALLLHAARPDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - protein, 512 aa, Artificial Sequence
  Synthetic polypeptide
MALPVTALLLPLALLLHAARPDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSAAAFVPVFLPAKPTTTPAPRPPTPAPTIASQPLSLRPEACRPAAGGAVHTRGLDFACDIYIWAPLAGTCGVLLLSLVITLYCNHRNRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - protein, 502 aa, Artificial Sequence
  Synthetic polypeptide
MALPVTALLLPLALLLHAARPDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSAAAFVPVFLPAKPTTTPAPRPPTPAPTIASQPLSLRPEACRPAAGGAVHTRGLDFACDIYIWAPLAGTCGVLLLSLVITLYCNHRNRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 12 - protein, 549 aa, Artificial Sequence
  Synthetic polypeptide
MALPVTALLLPLALLLHAARPDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSAAAFVPVFLPAKPTTTPAPRPPTPAPTIASQPLSLRPEACRPAAGGAVHTRGLDFACDIYIWAPLAGTCGVLLLSLVITLYCNHRNRFSVVKRGRKKLLYIFKQPFMRPVQTTQEEDGCSCRFPEEEEGGCELRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 13 - protein, 15 aa, Artificial Sequence
  Synthetic polypeptide
GGGGSGGGGSGGGGS

SEQ ID NO: 14 - protein, 18 aa, Artificial Sequence
  Synthetic polypeptide
GSTSGGGSGGGSGGGGSS

SEQ ID NO: 15 - protein, 18 aa, Artificial Sequence
  Synthetic polypeptide
GSTSGSGKPGSSEGSTKG

SEQ ID NO: 16 - protein, 8 aa, Artificial Sequence
  Synthetic polypeptide
GGGGSGGG

SEQ ID NO: 17 - protein, 16 aa, Artificial Sequence
  Synthetic polypeptide
VEPKSCDKTHTCPPCP

SEQ ID NO: 18 - protein, 16 aa, Artificial Sequence
  Synthetic polypeptide
LDPKSSDKTHTCPPCP

SEQ ID NO: 19 - protein, 16 aa, Artificial Sequence
  Synthetic polypeptide
VEPKSPDKTHTCPPCP

SEQ ID NO: 20 - protein, 11 aa, Artificial Sequence
  Synthetic polypeptide
LDKTHTCPPCP

SEQ ID NO: 21 - protein, 202 aa, Artificial Sequence
  Synthetic polypeptide
NKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 22 - protein, 27 aa, Artificial Sequence
  Synthetic polypeptide
FWVLVVVGGVLACYSLLVTVAFIIFWV

SEQ ID NO: 23 - protein, 109 aa, Artificial Sequence
  Synthetic polypeptide
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSAS

SEQ ID NO: 24 - protein, 48 aa, Artificial Sequence
  Synthetic polypeptide
AKPTTTPAPRPPTPAPTIASQPLSLRPEACRPAAGGAVHTRGLDFACD

SEQ ID NO: 25 - protein, 48 aa, Artificial Sequence
  Synthetic polypeptide
QRRKYRSNKGESPVEPAEPCHYSCPREEEGSTIPIQEDYRKPEPACSP

SEQ ID NO: 26 - protein, 112 aa, Artificial Sequence
  Synthetic polypeptide
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 27 - protein, 112 aa, Artificial Sequence
  Synthetic polypeptide
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 28 - protein, 41 aa, Artificial Sequence
  Synthetic polypeptide
RSKRSRLLHSDYMNMTPRRPGPTRKHYQPYAPPRDFAAYRS

SEQ ID NO: 29 - protein, 42 aa, Artificial Sequence
  Synthetic polypeptide
KRGRKKLLYIFKQPFMRPVQTTQEEDGCSCRFPEEEEGGCEL

SEQ ID NO: 30 - protein, 38 aa, Artificial Sequence
  Synthetic polypeptide
CWLTKKKYSSSVHDPNGEYMFMRAVNTAKKSRLTDVTL

SEQ ID NO: 31 - protein, 42 aa, Artificial Sequence
  Synthetic polypeptide
ALYLLRRDQRLPPDAHKPPGGGSFRTPIQEEQADAHSTLAKI

SEQ ID NO: 32 - protein, 48 aa, Artificial Sequence; identical to SEQ ID NO: 25
  Synthetic polypeptide

SEQ ID NO: 33 - protein, 194 aa, Artificial Sequence
  Synthetic polypeptide
KRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

(19) United States
(12) Patent Application Publication
(10) Pub. No.: US 2019/0030073 A1
(43) Pub. Date: Jan. 31, 2019

(54) CANCER IMMUNOTHERAPY WITH HIGHLY ENRICHED CD8+ CHIMERIC ANTIGEN RECEPTOR T CELLS

(71) Applicant: Cartesian Therapeutics, Inc., Rockville, MD (US)

(72) Inventors: Murat V. Kalayoglu, Silver Spring, MD (US); Metin Kurtoglu, Rockville, MD (US)

(73) Assignee: Cartesian Therapeutics, Inc., Rockville, MD (US)

(21) Appl. No.: 16/074,429

(22) PCT Filed: Dec. 1, 2017
(86) PCT No.: PCT/US17/64315

§ 371 Date: Jul. 31, 2018

Related U.S. Application Data
(60) Provisional application No. 62/429,661, filed on Dec. 2, 2016.

(51) Int. Cl. A61K 35/17; C07K 16/28; A61P 35/00

(52) U.S. Cl. CPC A61K 35/17; A61P 35/00; C07K 16/2878

(57) ABSTRACT

The invention provides a cellular immunotherapy therapy product comprising CAR T cells that are enriched in CD8+ cells. Also provided are methods for making and using the product.

### RELATED APPLICATIONS

This application claims priority under 35 U.S.C. § 119(e) to U.S. provisional patent application, U.S. Ser. No. 62/429,661, filed Dec. 2, 2016, the entire content of which is incorporated herein by reference.

### FIELD OF THE INVENTION

The present invention relates generally to the field of immuno-oncology, and more particularly to immune effector cells that are artificially modified to express a chimeric antigen receptor.

### BACKGROUND OF THE INVENTION

B cell malignancies are common hematological cancers that include multiple myeloma (MM), Hodgkin lymphoma (HL), non-Hodgkin lymphoma (NHL), chronic lymphocytic leukemia (CLL), and acute lymphoblastic leukemia (ALL). Traditional treatments for B cell malignancies, which include chemotherapy, radiotherapy and stem cell transplantation, have met with limited success due to toxicity, tumor resistance, incomplete tumor response, relapse, and secondary malignancies. Immune therapies with monoclonal antibodies also have shown limited success due in part to limited targeting and penetration at the tumor site.

A promising new approach to treating B cell malignancies is adoptive transfer of T cells genetically modified to recognize malignancy-associated antigens (see, e.g., Brenner et al., Current Opinion in Immunology 2010; 22:251-257; Rosenberg et al., Nature Reviews Cancer 2008; 8:299-308). T cells can be genetically modified by introduction of a nucleic acid construct to express chimeric antigen receptors (CARs), which are fusion proteins comprising an extracellular antigen recognition moiety and an intracellular T cell activation domain (see, e.g., Eshhar et al., Proc. Natl. Acad. Sci. USA, 1993; 90:720-724, and Sadelain et al., Curr. Opin. Immunol, 2009; 21:215-223. CAR T cells, such as those modified to recognize CD19, have shown benefit in treating B cell malignancies such as NHL and MM. However, such therapies have shown high toxicity, due in part to uncontrolled proliferation of the CAR T cells and secretion of inflammatory cytokines such as interferon gamma (IFN-γ). Therefore, there is a need for CAR T cell therapies that confer better safety and efficacy for the treatment of B cell malignancies.

### SUMMARY OF THE INVENTION

CD4+ and CD8+ T cells work in a coordinated fashion to mount immune responses. CAR T therapies known in the art comprise both CD4+ and CD8+ T cells. The achievement of anti-tumor effect is believed to require a combination CD4+ and CD8+ T cells. See, e.g., Turtle et al., J. Clin. Invest. 2016; 126:2123-2138. It is believed that an essential role of CD4+ cells is to secrete cytokines, e.g., interleukin-2, to maintain the survival and/or induce proliferation of CD8+ cells.

Paradoxically, the inventors have determined that products enriched in CD8+ T cells, e.g., wherein the T cells consist essentially of CD8+ cells, can confer significant advantages over products comprising both CD4+ and CD8+ cells. These products find particular use for the treatment of B cell malignancies, e.g., MM, as shown further herein.

T cells can be genetically modified by introduction of a nucleic acid, e.g., DNA or RNA, encoding a CAR. Generally, DNA is strongly preferred over RNA because DNA confers a permanent modification that is passed on to all clones during T cell clonal expansion, thereby multiplying the number of CAR T cells.

Paradoxically, for purposes of the present invention, the inventors have determined that the use of RNA, e.g., mRNA, can provide significant advantages over the use of DNA to modify a CD8+ cell to express a CAR. The use of RNA, e.g., mRNA, finds particular use for the treatment of B cell malignancies, e.g., MM, as shown further herein.

Thus, in one aspect, the invention provides a cell therapy product comprising CAR T cells directed against a B cell malignancy-associated antigen, wherein the T cells are highly enriched in CD8+ cells, e.g., at least 80% of the T cells in the cell therapy product are CD8+ cells, or alternatively, the T cells in the cell therapy product consist essentially of CD8+ cells. In this aspect of the invention, the concomitant use of a CAR-encoding mRNA construct can confer special advantages, as shown further herein.

In another aspect, the invention provides a method for producing a cell therapy product, the method comprising purifying CD8+ T cells and transfecting the cells with a nucleic acid construct encoding a CAR, whereby the resulting CD8+ CAR T cells are directed against a B cell malignancy-associated antigen. Also in this aspect of the invention, the concomitant use of a CAR-encoding mRNA construct can confer special advantages, as shown further herein.

In another aspect, the invention provides a method for producing a cell therapy product, the method comprising transfecting T cells with a nucleic acid construct encoding a CAR, whereby the resulting CAR T cells are directed against a B cell malignancy-associated antigen, and purifying CD8+ T cells from the CAR T cells. Also in this aspect of the invention, the concomitant use of a CAR-encoding mRNA construct can confer special advantages, as shown further herein.

In another aspect, the invention provides a method for treating a B cell malignancy in an individual, the method comprising administering to the individual a product or cell therapy according to the present invention.

In various aspects of the invention, further provided are CD8-enriched CAR T cells directed against B cell maturation antigen (BCMA; see, e.g., U.S. Patent Publication 2015-0051266, incorporated herein by reference). BCMA is selectively expressed in the B cell lineage, with the highest expression in plasma cells.

In various aspects of the invention, further provided are CAR T cells whereby the CAR-encoding nucleic acid is introduced by transfection, e.g., by electroporation.

The invention can be further understood by reference to the description, embodiments, and examples below.

### BRIEF DESCRIPTION OF THE DRAWINGS

FIG. 1 shows a flow cytometry scatterplot showing CD8 cells populations isolated by CD3 magnetic beads vs CD8 magnetic beads.

FIG. 2 shows a flow cytometry scatterplot showing BCMA-CAR expression and viability of transfected CD8+ T-cells.

FIG. 3 shows a flow cytometry histogram showing cytotoxicity of RPMI-8226 myeloma tumor cell line following coincubation with untransfected or transfected T cells.

FIG. 4 shows a fluorescent photomicrograph showing BCMA CAR-transfected CD8+ T cells directly kill RPMI-8226 tumor cells. 400× magnification. Green indicates calcein AM (live) stained RPMI-8226 cells. Red indicates propidium iodide (dead) stained cells.

FIG. 5 provides a graph showing levels of cell activation (degranulation), as measured by LAMP1-positivity, for CD3+ or CD8+ CAR T-cells.

FIG. 6 provides a graph showing the time course of myeloma tumor growth, as measured by tumor bioluminescence in immunodeficient mice.

FIG. 7 shows a graph of serum interferon gamma (IFN) concentrations in immunodeficient mice that have myeloma tumors. The graph compares mice treated with enriched CD8+ CAR T-cells versus mixed CD4+/CD8+(CD3+) CAR T-cells.

FIG. 8 is a graph showing cell viability (top) and the relative expression of CAR RNA transfection into CD8+ versus CD4+ human T cells.

### DEFINITIONS

As used herein, “nucleic acid sequence” is intended to encompass a polymer of DNA or RNA, i.e., a polynucleotide, which can be single-stranded or double-stranded and which can contain non-natural or altered nucleotides. The terms “nucleic acid” and “polynucleotide” as used herein refer to a polymeric form of nucleotides of any length, either ribonucleotides (RNA) or deoxyribonucleotides (DNA). These terms refer to the primary structure of the molecule, and thus include double- and single-stranded DNA, and double- and single-stranded RNA. The terms include, as equivalents, analogs of either RNA or DNA made from nucleotide analogs and modified polynucleotides such as, though not limited to methylated and/or capped polynucleotides (e.g., CleanCap from Trilink Biotechnologies, as well as ARCA, mCAP, Cap-0, Cap-1, and Cap-2). “mRNA” refers to messenger RNA. It should be appreciated that an RNA or mRNA can contain natural and/or unnatural nucleotides (e.g., ribonucleotides). Exemplary RNA molecules, e.g., modified RNA molecules are provided herein.

As used herein, “synthetic nucleic acid construct” refers to a nucleic acid sequence that does not occur in nature. For the present invention, the synthetic nucleic acid construct is preferably adapted to express one or more proteins, e.g., a CAR, in a T cell.

As used herein, “chimeric antigen receptor” or “CAR” refers to a fusion protein comprising an extracellular antigen recognition moiety and an intracellular T cell activation domain.

As used herein with reference to a CAR, “express” and “expression” mean that a cell produces a CAR protein, for example, that is capable if generating a signal within the cell.

As used herein, a “B cell malignancy” refers to a tumor principally comprised of B cells or lymphopoietic precursors thereof. A B cell malignancy can include, for example, MM, HL, NHL, CLL, and ALL.

As used herein, a “B cell malignancy-associated antigen” refers to an antigen that is typically found on a B cell malignancy but not found, or less typically found, on normal host cells. B cell malignancy-associated antigens include CD19 and BCMA.

As used herein with reference to antigen recognition, “to bind” refers to an attractive interaction between an antigen and antigen recognition moiety that is sufficient to induce T cell activation.

As used herein, a “carrier” is any material or medium used to hold, transfer, stabilize, or deliver the other components of a cell therapy product.

As used herein, “transfect,” “transfection,” “transfected,” and “transfecting” refer to a process whereby a nucleic acid is deliberately introduced into a cell without the use of a viral vector, e.g., physical, electrical, and chemical based methods, e.g., electroporation (including nucleofection), cell squeezing, sonoporation, optical transfection, calcium phosphate transfection, and particle-based methods. Exemplary particle-based methods include, without limitation, precious-metal-based, liposomal, polymer-based, or endosome-based nanoparticles. Polymer-based nanoparticles may include, for example, poly[beta]-amino esters or other chemicals with biodegradable and pH-sensitive properties. Nanoparticles may be coated with, for example, polyglutamic acid (PGA) and/or antibodies or antibody-fragments targeting cell-membrane antigens, for example CD4, CD8, CD3, CD56, to facilitate uptake.

As used herein, “electroporation” includes any process whereby an electric current is applied to a cell for the purpose of introducing a nucleic acid into the cell.

As used herein, “transduce,” “transduction,” “transduced,” and “transducing” refer a process whereby a nucleic acid is deliberately introduced into a cell by use of a viral vector.

As used herein with reference to CD8+ cells, “enrich,” “enriched,” “enrichment,” “purify,” “purified,” and “purification” are used interchangeably to mean that a first sample of CD8+ cells is processed, e.g., by cell sorting, positive selection, or negative selection, to obtain a second sample that has a higher proportion of CD8+ cells, as compared to the first sample. The proportion of CD8+ cells (“CD8+ Proportion”) is the number of CD8+ cells divided by the total number of T cells in a sample, except where the context indicates otherwise. The CD8+ Proportion can optionally be expressed as a percentage. The CD8+ Proportion can indicate the degree of enrichment or purity of a sample or product. For practical purposes, the CD8+ Proportion can be approximated, for example, as:

[CD8]/([CD8]+[CD4])

or

[CD8]/[CD3],

where [x] refers to the number of cells of the respective cell type. For practical purposes, the CD8+ Proportion can be estimated from any measurement known to suitably correlate with the number of CD8+ cells and CD3+, CD4+, and/or T cells, e.g., volumetric or immunoreactive signal measurements.

“Purity” and “highly enriched” are relative terms and are not intended to mean absolute purity. It is to be understood that CD8+ cells may be provided or formulated or diluted with cellular or non-cellular constituents, e.g., carriers, and still for practical purposes be purified or enriched.

As used herein, the term “highly enriched” or “highly purified” means that at least 80 percent of the T cells in a product are CD8+ cells.

As used herein with reference to a sample, product, or other plurality of T cells comprising CD8+ cells, “mixed” means that more than 20 percent of the T cells are not CD8+ cells, e.g, more than 20 percent of the T cells are CD4+.

As used herein, the term “monoclonal antibody” refers to an antibody that is produced by a single clone of B cells and binds to the same epitope. The antigen recognition moiety of the CAR encoded by a nucleic acid sequence can be a whole antibody or an antibody fragment. A whole antibody typically consists of four polypeptides: two identical copies of a heavy (H) chain polypeptide and two identical copies of a light (L) chain polypeptide. Each of the heavy chains contains one N-terminal variable (VH) region and three C-terminal constant (CH1, CH2 and CH3) regions, and each light chain contains one N-terminal variable (VL) region and one C-terminal constant (CL) region. The variable regions of each pair of light and heavy chains form the antigen binding site of an antibody. The VH and VL regions have the same general structure, with each region comprising four framework regions, whose sequences are relatively conserved. The framework regions are connected by three complementarity determining regions (CDRs). The three CDRs, known as CDR1, CDR2, and CDR3, form the “hypervariable region” of an antibody, which is responsible for antigen binding.

As used herein, the term “antigen recognition moiety,” refers to one or more fragments or portions of an antibody that retain the ability to specifically bind to an antigen (see, generally, Holliger et al., Nat. Biotech. 2005; 23:1126-1129.

As used herein “suffer,” “suffers,” or “suffering” from refers to an individual diagnosed with a particular disease or condition.

As used herein, unless otherwise specified, the terms “treat,” “treating,” and “treatment” contemplate an action that occurs while an individual is suffering from the specified disease or condition, which cures or reduces the severity of the disease of condition, or slows the progression of the disease or condition.

As used herein, “first-line therapy” refers to a therapy that is suitable or desirable for use in an individual concomitant with or prior to the use of other therapies.

### DETAILED DESCRIPTION OF CERTAIN EMBODIMENTS OF THE INVENTION

### Cell Therapy Product

In one aspect, the invention provides a cell therapy product comprising: a plurality of T cells, wherein at least 80 percent of the T cells of the plurality are CD8+ cells, wherein at least some (e.g., at least 20%, 30%, 40%, 50%, 60%, 70%, 80%, 90%, or 95%) of the CD8+ cells express a CAR protein, wherein the protein comprises a CAR (e.g., a non-naturally occurring CAR). In some embodiments, the invention provides a cell therapy product comprising: a plurality of T cells, wherein at least 80 percent of the T cells are CD8+ cells, wherein at least some (e.g., at least 20%, 30%, 40%, 50%, 60%, 70%, 80%, 90%, or 95%) of the CD8+ cells express a CAR protein, wherein the protein comprises an antigen recognition moiety and a T cell activation moiety, and wherein the antigen recognition moiety binds to a B cell malignancy-associated antigen. In some embodiments, the CAR protein further comprises a transmembrane domain. In some embodiments, CAR protein comprises, arranged from extracellular to intracellular: an antigen recognition moiety, a transmembrane domain, and a T cell activation moietry. It should be appreciated that the cell therapy product may comprise T cells expressing any of the CARs provided herein.

In some embodiments, the cell therapy product is essentially free of CD4+ cells. In some embodiments, less than 20%, less than 15%, less than 10%, less than 7%, less than 5%, less than 3%, less than 2%, less than 1%, less than 0.5%, or less than 0.1% of the T cells in the cell therapy product are CD4+ cells.

In some embodiments, the T cells in the cell therapy product consist essentially of CD8+ cells. In some embodiments, at least 80 percent, at least 85 percent, at least 90 percent, at least 93 percent, at least 95 percent, at least 95 percent, at least 97 percent, at least 98 percent, at least 99 percent, at least 99.5 percent, or at least 99.9 percent of the T cells in the cell therapy product are CD8+ cells. In some embodiments, at least 85 percent of the T cells in the cell therapy product are CD8+ cells. In some embodiments, at least 90 percent of the T cells in the cell therapy product are CD8+ cells. In some embodiments, at least 93 percent of the T cells in the cell therapy product are CD8+ cells. In some embodiments, at least 95 percent of the T cells in the cell therapy product are CD8+ cells. In some embodiments, at least 97 percent of the T cells in the cell therapy product are CD8+ cells. In some embodiments, at least 98 percent of the T cells in the cell therapy product are CD8+ cells. In some embodiments, at least 99 percent of the T cells in the cell therapy product are CD8+ cells. In some embodiments, at least 99.5 percent of the T cells in the cell therapy product are CD8+ cells. In some embodiments, at least 99.9 percent of the T cells in the cell therapy product are CD8+ cells.

In some embodiments, at least 80 percent of the CD8+ cells express the CAR (e.g., any of the CARs provided herein). In some embodiments, at least 90 percent of the CD8+ cells express the CAR. In some embodiments, at least 95 percent of the CD8+ cells express the CAR. In some embodiments, at least 97 percent of the CD8+ cells express the CAR. In some embodiments, at least 98 percent of the CD8+ cells express the CAR. In some embodiments, at least 99 percent of the CD8+ cells express the CAR. In some embodiments, at least 99.5 percent of the CD8+ cells express the CAR. In some embodiments, at least 99.9 percent of the CD8+ cells express the CAR.

In some embodiments, at least some (e.g., at least 10%, 20%, 30%, 40%, 50%, 60%, 70%, 80%, 90%, or 95%) of the CD8+ cells comprise a nucleic acid construct that encodes the CAR. In some embodiments, at least some of the CD8+ cells comprise an RNA (e.g., mRNA) that encodes the CAR. In some embodiments, at least some (e.g., at least 10%, 20%, 30%, 40%, 50%, 60%, 70%, 80%, 90%, or 95%) of the CD8+ cells comprise mRNA that encodes the CAR. In some embodiments, at least some (e.g., at least 10%, 20%, 30%, 40%, 50%, 60%, 70%, 80%, 90%, or 95%) of the CD8+ cells comprise DNA that encodes the CAR. In some embodiments, the RNA, mRNA, or DNA comprises one or more unnatural nucleotides. In some embodiments, the RNA, mRNA, or DNA is synthetic.

Nucleotides (e.g., RNA polynucleotides, such as mRNA polynucleotides), in some embodiments, comprise various (more than one) different modifications. In some embodiments, a particular region of a polynucleotide contains one, two or more (optionally different) nucleoside or nucleotide modifications. In some embodiments, a modified RNA polynucleotide (e.g., a modified mRNA polynucleotide), introduced to a cell or organism, exhibits reduced degradation in the cell or organism, respectively, relative to an unmodified polynucleotide. In some embodiments, a modified RNA polynucleotide (e.g., a modified mRNA polynucleotide), introduced into a cell or organism, may exhibit reduced immunogenicity in the cell or organism, respectively (e.g., a reduced innate response).

Modifications of polynucleotides include, without limitation, those described herein. Polynucleotides (e.g., RNA polynucleotides, such as mRNA polynucleotides) may comprise modifications that are naturally-occurring, non-naturally-occurring or the polynucleotide may comprise a combination of naturally-occurring and non-naturally-occurring modifications. Polynucleotides may include any useful modification, for example, of a sugar, a nucleobase, or an internucleoside linkage (e.g., to a linking phosphate, to a phosphodiester linkage or to the phosphodiester backbone).

Polynucleotides (e.g., RNA polynucleotides, such as mRNA polynucleotides), in some embodiments, comprise non-natural modified nucleotides that are introduced during synthesis or post-synthesis of the polynucleotides to achieve desired functions or properties. The modifications may be present on an internucleotide linkages, purine or pyrimidine bases, or sugars. The modification may be introduced with chemical synthesis or with a polymerase enzyme at the terminal of a chain or anywhere else in the chain. Any of the regions of a polynucleotide may be chemically modified.

The present disclosure provides for modified nucleosides and nucleotides of a polynucleotide (e.g., RNA polynucleotides, such as mRNA polynucleotides). A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A nucleotide” refers to a nucleoside, including a phosphate group. Modified nucleotides may by synthesized by any useful method, such as, for example, chemically, enzymatically, or recombinantly, to include one or more modified or non-natural nucleosides. Polynucleotides may comprise a region or regions of linked nucleosides. Such regions may have variable backbone linkages. The linkages may be standard phosphdioester linkages, in which case the polynucleotides would comprise regions of nucleotides.

Modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures. One example of such non-standard base pairing is the base pairing between the modified nucleotide inosine and adenine, cytosine or uracil. Any combination of base/sugar or linker may be incorporated into polynucleotides of the present disclosure.

In some embodiments, the T cells in the cell therapy product consist essentially of CD8+ cells that express the CAR. In some embodiments, the cells in the cell therapy product consist essentially of CD8+ cells that express the CAR.

In some embodiments, the CD8+ cells have been modified to express a CAR by introduction of RNA. In some embodiments, the CD8+ have been modified to express a CAR by introduction of mRNA.

In some embodiments, the CD8+ cells express two or more CARs, i.e., two or more CARs wherein the respective amino acid sequences differ deliberately by at least one amino acid. In some embodiments, the CD8+ cells express two or more CARs that each bind the same B cell malignancy-associated antigen. In some embodiments, the CD8+ cells express two or more CARs that each bind different B cell malignancy-associated antigens. In some embodiments, the CD8+ cells express two or more CARs, at least one of which binds to a B cell malignancy-associated antigen, and at least one of which does not bind to B cell malignancy-associated antigens. In some embodiments, the CD8+ cells express two CARs that each bind BCMA. In some embodiments, the CD8+ cells express two CARs, of which one binds BCMA and the other binds a B cell malignancy-associated antigen that is not BCMA, e.g., CS1 and/or CD38. In some embodiments, the CD8+ cells express two CARs, of which one binds BCMA and the other binds an antigen that is not B cell malignancy-associated antigen.

Methods for engineering T cells and for enriching for T cells (e.g., CD8+ T cells) would be apparent to the skilled artisan and are described in further detail herein.

In some embodiments, the product is a final product suitable for administration to humans. In some embodiments, the product further comprises a carrier (e.g., a pharmaceutically acceptable carrier).

In some embodiments, the T cell activation domain comprises a domain selected from the group consisting of: a human CD8-alpha protein, a human CD28 protein, a human CD3-zeta protein, a human FcRγ protein, a CD27 protein, an OX40 protein, a human 4-1BB protein, and modified version any of the foregoing. In some embodiments, the T cell activation domain is a human CD3-zeta protein.

In some embodiments, the B cell malignancy-associated antigen is selected from the group consisting of BCMA, CD19, CS1, CD38, CD138, CD30, CD20, and CD25. In some embodiments, the B cell malignancy-associated antigen is BCMA. In some embodiments, the B cell malignancy-associated antigen is CD19. In some embodiments, the B cell malignancy-associated antigen is CS1. In some embodiments, the B cell malignancy-associated antigen is CD38. In some embodiments, the B cell malignancy-associated antigen is CD138. In some embodiments, the B cell malignancy-associated antigen is CD30. In some embodiments, the B cell malignancy-associated antigen is CD20. In some embodiments, the B cell malignancy-associated antigen is CD25.

In some embodiments, the antigen recognition moiety comprises a variable region of an antibody (e.g., a monoclonal antibody), which can be engineered into other formats, e.g. a scFV format. In some embodiments, the antigen recognition moiety comprises the (i) heavy chain complementarity determining region (CDR)1, (ii) heavy chain CDR2, (iii) heavy chain CDR3, (iv) light chain CDR1, (v) light chain CDR2, and (vi) light chain CDR3 of any of the scFV amino acid sequences from the CARs selected from the group consisting of SEQ ID NO: 4, SEQ ID NO: 5, SEQ ID NO: 6, SEQ ID NO: 8, SEQ ID NO: 9, SEQ ID NO: 10, SEQ ID NO: 11, and SEQ ID NO: 12. In some embodiments, the antigen recognition moiety comprises the (i) heavy chain variable region and (ii) light chain variable region of any one of any scFV amino acid sequences from the CARs selected from the group consisting of SEQ ID NO: 4, SEQ ID NO: 5, SEQ ID NO: 6, SEQ ID NO: 8, SEQ ID NO: 9, SEQ ID NO: 10, SEQ ID NO: 11, and SEQ ID NO: 12. In some embodiments, the CAR comprises the amino acid sequence of any of the amino acid sequences selected from the group consisting of SEQ ID NO: 4, SEQ ID NO: 5, SEQ ID NO: 6, SEQ ID NO: 8, SEQ ID NO: 9, SEQ ID NO: 10, SEQ ID NO: 11, and SEQ ID NO: 12. In some embodiments, the CAR binds BCMA. Exemplary CARs that bind BCMA have been described previously, for example, in patent application U.S. Ser. No. 14/389,677, which published as US 2015/0051266 on Feb. 19, 2015; the entire contents of which is incorporated herein by reference.

Exemplary CARs that bind BCMA are provided below (the cytoplasmic CD3-zeta portion is indicated by underlining; the spacer sequence linking the variable heavy and variable light chains of the scFV are shown in bold):

(SEQ ID NO: 4)
MALPVTALLLPLALLLHAARPDIVLTQSPPSLAMSLGKRATISCRASESVTILGSHLIY
WYQQKPGQPPTLLIQLASNVQTGVPARFSGSGSRTDFTLTIDPVEEDDVAVYYCLQS
RTIPRTFGGGTKLEIKGSTSGSGKPGSGEGSTKGQIQLVQSGPELKKPGETVKISCKA
SGYTFRHYSMNWVKQAPGKGLKWMGRINTESGVPIYADDFKGRFAFSVETSASTAY
LVINNLKDEDTASYFCSNDYLYSLDFWGQGTALTVSSFVPVFLPAKPTTTPAPRPPTP
APTIASQPLSLRPEACRPAAGGAVHTRGLDFACDIYIWAPLAGTCGVLLLSLVITLYC
NHRNRSKRSRLLHSDYMNMTPRRPGPTRKHYQPYAPPRDFAAYRSRVKFSRSADAP
AYQQGQNQLYNELNLGRREEYDVLDKRRGRDPEMGGKPRRKNPQEGLYNELQKD
KMAEAYSEIGMKGERRRGKGHDGLYQGLSTATKDTYDALHMQALPPR
(SEQ ID NO: 5)
MALPVTALLLPLALLLHAARPDIVLTQSPPSLAMSLGKRATISCRASESVTILGSHLIH
WYQQKPGQPPTLLIQLASNVQTGVPARFSGSGSRTDFTLTIDPVEEDDVAVYYCLQS
RTIPRTFGGGTKLEIKGSTSGSGKPGSGEGSTKGQIQLVQSGPELKKPGETVKISCKA
SGYTFTDYSINWVKRAPGKGLKWMGWINTETREPAYAYDFRGRFAFSLETSASTAY
LQINNLKYEDTATYFCALDYSYAMDYWGQGTSVTVSSFVPVFLPAKPTTTPAPRPPT
PAPTIASQPLSLRPEACRPAAGGAVHTRGLDFACDIYIWAPLAGTCGVLLLSLVITLY
CNHRNRSKRSRLLHSDYMNMTPRRPGPTRKHYQPYAPPRDFAAYRSRVKFSRSADA
PAYQQGQNQLYNELNLGRREEYDVLDKRRGRDPEMGGKPRRKNPQEGLYNELQKD
KMAEAYSEIGMKGERRRGKGHDGLYQGLSTATKDTYDALHMQALPPR
(SEQ ID NO: 6)
MALPVTALLLPLALLLHAARPDIVLTQSPPSLAMSLGKRATISCRASESVTILGSHLIY
WYQQKPGQPPTLLIQLASNVQTGVPARFSGSGSRTDFTLTIDPVEEDDVAVYYCLQS
RTIPRTFGGGTKLEIKGSTSGSGKPGSGEGSTKGQIQLVQSGPELKKPGETVKISCKA
SGYTFTHYSMNWVKQAPGKGLKWMGRINTETGEPLYADDFKGRFAFSLETSASTAY
LVINNLKNEDTATFFCSNDYLYSCDYWGQGTTLTVSSFVPVFLPAKPTTTPAPRPPTP
APTIASQPLSLRPEACRPAAGGAVHTRGLDFACDIYIWAPLAGTCGVLLLSLVITLYC
NHRNRSKRSRLLHSDYMNMTPRRPGPTRKHYQPYAPPRDFAAYRSRVKFSRSADAP
AYQQGQNQLYNELNLGRREEYDVLDKRRGRDPEMGGKPRRKNPQEGLYNELQKD
KMAEAYSEIGMKGERRRGKGHDGLYQGLSTATKDTYDALHMQALPPR
(SEQ ID NO: 8)
MLLLVTSLLLCELPHPAFLLIPDIVLTQSPPSLAMSLGKRATISCRASESVTILGSHLIH
WYQQKPGQPPTLLIQLASNVQTGVPARFSGSGSRTDFTLTIDPVEEDDVAVYYCLQS
RTIPRTFGGGTKLEIKGSTSGSGKPGSGEGSTKGQIQLVQSGPELKKPGETVKISCKA
SGYTFTDYSINWVKRAPGKGLKWMGWINTETREPAYAYDFRGRFAFSLETSASTAY
LQINNLKYEDTATYFCALDYSYAMDYWGQGTSVTVSSAAAFVPVFLPAKPTTTPAP
RPPTPAPTIASQPLSLRPEACRPAAGGAVHTRGLDFACDIYIWAPLAGTCGVLLLSLVI
TLYCNHRNRSKRSRLLHSDYMNMTPRRPGPTRKHYQPYAPPRDFAAYRSRVKFSRS
ADAPAYQQGQNQLYNELNLGRREEYDVLDKRRGRDPEMGGKPRRKNPQEGLYNEL
QKDKMAEAYSEIGMKGERRRGKGHDGLYQGLSTATKDTYDALHMQALPPR
(SEQ ID NO: 9)
MALPVTALLLPLALLLHAARPDIVLTQSPPSLAMSLGKRATISCRASESVTILGSHLIH
WYQQKPGQPPTLLIQLASNVQTGVPARFSGSGSRTDFTLTIDPVEEDDVAVYYCLQS
RTIPRTFGGGTKLEIKGSTSGSGKPGSGEGSTKGQIQLVQSGPELKKPGETVKISCKA
SGYTFTDYSINWVKRAPGKGLKWMGWINTETREPAYAYDFRGRFAFSLETSASTAY
LQINNLKYEDTATYFCALDYSYAMDYWGQGTSVTVSSAAAFVPVFLPAKPTTTPAP
RPPTPAPTIASQPLSLRPEACRPAAGGAVHTRGLDFACDIYIWAPLAGTCGVLLLSLVI
TLYCNHRNRSKRSRLLHSDYMNMTPRRPGPTRKHYQPYAPPRDFAAYRSRVKFSRS
ADAPAYQQGQNQLYNELNLGRREEYDVLDKRRGRDPEMGGKPRRKNPQEGLYNEL
QKDKMAEAYSEIGMKGERRRGKGHDGLYQGLSTATKDTYDALHMQALPPR
(SEQ ID NO: 10)
MALPVTALLLPLALLLHAARPDIVLTQSPPSLAMSLGKRATISCRASESVTILGSHLIH
WYQQKPGQPPTLLIQLASNVQTGVPARFSGSGSRTDFTLTIDPVEEDDVAVYYCLQS
RTIPRTFGGGTKLEIKGSTSGSGKPGSGEGSTKGQIQLVQSGPELKKPGETVKISCKA
SGYTFTDYSINWVKRAPGKGLKWMGWINTETREPAYAYDFRGRFAFSLETSASTAY
LQINNLKYEDTATYFCALDYSYAMDYWGQGTSVTVSSAAAFVPVFLPAKPTTTPAP
RPPTPAPTIASQPLSLRPEACRPAAGGAVHTRGLDFACDIYIWAPLAGTCGVLLLSLVI
TLYCNHRNRFSVVKRGRKKLLYIFKQPFMRPVQTTQEEDGCSCRFPEEEEGGCELRV
KFSRSADAPAYQQGQNQLYNELNLGRREEYDVLDKRRGRDPEMGGKPRRKNPQEG
LYNELQKDKMAEAYSEIGMKGERRRGKGHDGLYQGLSTATKDTYDALHMQALPPR
(SEQ ID NO: 11)
MALPVTALLLPLALLLHAARPDIVLTQSPPSLAMSLGKRATISCRASESVTILGSHLIH
WYQQKPGQPPTLLIQLASNVQTGVPARFSGSGSRTDFTLTIDPVEEDDVAVYYCLQS
RTIPRTFGGGTKLEIKGSTSGSGKPGSGEGSTKGQIQLVQSGPELKKPGETVKISCKA
SGYTFTDYSINWVKRAPGKGLKWMGWINTETREPAYAYDFRGRFAFSLETSASTAY
LQINNLKYEDTATYFCALDYSYAMDYWGQGTSVTVSSAAAFVPVFLPAKPTTTPAP
RPPTPAPTIASQPLSLRPEACRPAAGGAVHTRGLDFACDIYIWAPLAGTCGVLLLSLVI
TLYCNHRNRRDQRLPPDAHKPPGGGSFRTPIQEEQADAHSTLAKIRVKFSRSADAPA
YQQGQNQLYNELNLGRREEYDVLDKRRGRDPEMGGKPRRKNPQEGLYNELQKDK
MAEAYSEIGMKGERRRGKGHDGLYQGLSTATKDTYDALHMQALPPR
(SEQ ID NO: 12)
MALPVTALLLPLALLLHAARPDIVLTQSPPSLAMSLGKRATISCRASESVTILGSHLIH
WYQQKPGQPPTLLIQLASNVQTGVPARFSGSGSRTDFTLTIDPVEEDDVAVYYCLQS
RTIPRTFGGGTKLEIKGSTSGSGKPGSGEGSTKGQIQLVQSGPELKKPGETVKISCKA
SGYTFTDYSINWVKRAPGKGLKWMGWINTETREPAYAYDFRGRFAFSLETSASTAY
LQINNLKYEDTATYFCALDYSYAMDYWGQGTSVTVSSAAAFVPVFLPAKPTTTPAP
RPPTPAPTIASQPLSLRPEACRPAAGGAVHTRGLDFACDIYIWAPLAGTCGVLLLSLVI
TLYCNHRNRFSVVKRGRKKLLYIFKQPFMRPVQTTQEEDGCSCRFPEEEEGGCELRR
DQRLPPDAHKPPGGGSFRTPIQEEQADAHSTLAKIRVKFSRSADAPAYQQGQNQLYN
ELNLGRREEYDVLDKRRGRDPEMGGKPRRKNPQEGLYNELQKDKMAEAYSEIGMK
GERRRGKGHDGLYQGLSTATKDTYDALHMQALPPR

In some embodiments, the CAR comprises an antigen recognition moiety that binds to BCMA. In some embodiments, the CAR is expressed in a CD8+ cell by introduction of mRNA encoding the CAR.

In some embodiments, the CAR comprises an antigen recognition moiety that binds to BCMA and a T cell activation domain comprising a human CD3-zeta protein. In some embodiments, the nucleic acid construct comprises DNA that encodes a CAR comprising: an antigen recognition moiety that binds to BCMA; and a T cell activation domain comprising a human CD3-zeta protein.

In some embodiments, the antibody recognition moiety comprises a single-domain antibody, a camelid heavy-chain antibody, IgNAR, Fab fragments, Fab′ fragments, F(ab)′2 fragments, F(ab)′3 fragments, Fv, single 20 chain Fv proteins (“scFv”), bis-scFv, minibodies, diabodies, triabodies, tetrabodies, disulfide stabilized Fv proteins (“dsFv”), and single-domain antibody (sdAb, Nanobody) and portions of full length antibodies responsible for antigen binding. The term also includes genetically engineered forms such as chimeric antibodies (for example, humanized murine antibodies), heteroconjugate antibodies (e.g., bispecific antibodies) and antigen binding fragments thereof.

In some embodiments, the antibody recognition moiety comprises a centyrin.

In some embodiments, the CAR comprises the amino acid sequence of SEQ ID NO: 5. In some embodiments, the CAR comprising the amino acid sequence of SEQ ID NO: 5 is encoded by a nucleic acid construct that is DNA. In some embodiments, the CAR comprising the amino acid sequence of SEQ ID NO: 5 is encoded by a nucleic acid construct that is RNA. In some embodiments, the CAR comprising the amino acid sequence of SEQ ID NO: 5 is encoded by a nucleic acid construct that is mRNA.

Methods to prepare a nucleic acid construct comprising a specified nucleotide sequence are generally known in the art.

In some embodiments, at least 80 percent, at least 90 percent, at least 93 percent, at least 95 percent, at least 95 percent, at least 97 percent, at least 98 percent, at least 99 percent, at least 99.5 percent, or at least 99.9 percent of the T cells in the cell therapy product express a CAR comprising an antigen recognition moiety that binds to BCMA; and a T cell activation domain comprising a human CD3-zeta protein; and in any of the embodiments in this paragraph, the CAR can be encoded by a nucleic acid construct that is mRNA. In any of the embodiments in this paragraph, the nucleic acid construct can be introduced into the T cell by electroporation.

In some embodiments, at least 80 percent, at least 90 percent, at least 93 percent, at least 95 percent, at least 95 percent, at least 97 percent, at least 98 percent, at least 99 percent, at least 99.5 percent, or at least 99.9 percent of the T cells in the cell therapy product are CD8+ cells and express a CAR comprising an antigen recognition moiety that binds to BCMA; and a T cell activation domain comprising a human CD3-zeta protein; and in any of the embodiments in this paragraph, the CAR can be encoded by a nucleic acid construct that is mRNA. In any of the embodiments in this paragraph, the nucleic acid construct can be introduced into the T cell by electroporation.

In some embodiments, at least 80 percent, at least 90 percent, at least 93 percent, at least 95 percent, at least 95 percent, at least 97 percent, at least 98 percent, at least 99 percent, at least 99.5 percent, or at least 99.9 percent of the T cells in the cell therapy product are CD8+ cells and express a CAR comprising the amino acid sequences of SEQ ID NO: 5; and in any of the embodiments in this paragraph, the CAR can be encoded by a nucleic acid construct that is mRNA. In any of the embodiments in this paragraph, the nucleic acid construct can be introduced into the T cell by electroporation.

### Methods for Producing a Cell Therapy Product

In another aspect, the invention provides a method for producing a cell therapy product, the method comprising purifying CD8+ T cells and transfecting the cells with a nucleic acid construct encoding a CAR, whereby the resulting CD8+ CAR T cells are directed against a B cell malignancy-associated antigen.

In another aspect, the invention provides a method for producing a cell therapy product, the method comprising transfecting T cells with a nucleic acid construct encoding a CAR, whereby the resulting CAR T cells are directed against a B cell malignancy-associated antigen, and purifying CD8+ T cells from the CAR T cells.

Methods for enriching or purifying CD8+ cells, e.g., separating CD8+ cells from CD4+ cells, are generally known in the art. In some embodiments, the CD8+ cells are purified by cell sorting. In some embodiments, the CD8+ cells are purified by positive selection. Positive selection can be carried out, for example, by use of antibodies or other CD8- or CD8/CD28-specific binding molecules, which may optionally be coated on paramagnetic beads. In some embodiments, the CD8+ cells are purified by negative selection. Negative selection can be carried out, for example, by expanding peripheral blood mononuclear cells with antibodies directed against non-CD8 cells, for example an anti-CD4 antibody with or without an anti-CD14 antibody.

In some embodiments, the CD8 cells are transfected or transduced 0, about 0, 2, about 2, 4, about 4, 8, about 8, 12, about 12, 24, or about 24 hours after being enriched or purified. In some embodiments, the CD8 cells are activated, for example by addition of interleukin-2 and/or interleukin-15, about 0, 2, about 2, 4, about 4, 8, about 8, 12, about 12, 24, or about 24 hours after transfection or transduction. In some embodiments, the cell therapy product produced by the method is a final product suitable for human administration. In some embodiments, the transfected or transduced CD8+ cells are cryopreserved.

In some embodiments, the nucleic acid construct is introduced into the CD8+ cell or T cell by transfection. In some embodiments, the transfection comprises electroporation, nucleofection, cell squeezing, sonoporation, optical transfection, calcium phosphate transfection, and/or particle-based delivery.

In another aspect, the invention provides a method for producing a cell therapy product, the method comprising purifying CD8+ T cells and transducing the cells with a nucleic acid construct encoding a CAR, whereby the resulting CD8+ CAR T cells are directed against a B cell malignancy-associated antigen.

In another aspect, the invention provides a method for producing a cell therapy product, the method comprising transducing T cells with a nucleic acid construct encoding a CAR, whereby the resulting CAR T cells are directed against a B cell malignancy-associated antigen, and purifying CD8+ T cells from the CAR T cells. In some embodiments of this aspect of the invention, the nucleic acid construct further encodes a marker or enzyme useful for purifying CD8+ T cells and/or CAR T cells, e.g., beta-galactosidase, luciferase, and/or similar proteins known in the art. In some embodiments of this aspect of the invention, a second nucleic acid construct that encodes a marker or enzyme useful for purifying CD8+ T cells and/or CAR T cells, e.g., beta-galactosidase, luciferase, and/or similar proteins known in the art, is introduced into the T cell concomitantly with the nucleic acid construct encoding the CAR.

In some embodiments, the nucleic acid construct is introduced into the CD8+ cell or T cell by viral transduction. In some embodiments, CD8+ cells are transduced with a CAR-encoding viral vector. The construction of such vectors is generally known in the art. The viral vector can be, for example, gamma-retroviral vector or lentiviral vector. The CD8 cells can be transduced, for example by incubating the vector with CD8 cells. In some embodiments, the process of transduction is performed more than once on the same cells. In some embodiments of this aspect of the invention, the nucleic acid construct further encodes a marker or enzyme useful for purifying CD8+ T cells and/or CAR T cells, e.g., beta-galactosidase, luciferase, and/or similar proteins known in the art. In some embodiments of this aspect of the invention, a second nucleic acid construct that encodes a marker or enzyme useful for purifying CD8+ T cells and/or CAR T cells, e.g., beta-galactosidase, luciferase, and/or similar proteins known in the art, is introduced into the T cell concomitantly with the nucleic acid construct encoding the CAR.

In some embodiments, the method is adapted to produce any of the embodiments described under “Cell Therapy Product,” supra.

### Chimeric Antigen Receptors

Some aspects of the disclosure provide chimeric antigen receptors that bind to a B cell malignancy-associated antigen (e.g., BCMA, CD19, CS1, CD38, CD138, CD30, CD20, or CD25). In some embodiments, the a chimeric antigen receptor (CAR) comprises (a) an extracellular domain comprising an antigen binding domain, (b) a transmembrane domain and (c) a cytoplasmic domain. It should be appreciated that in some embodiments, CAR molecules described by the following exemplary, non-limiting arrangements are from left to right, N-terminus to C-terminus of the CAR. A CAR molecule as described by the disclosure may comprise or further comprise any other combination of elements as described herein.

In some embodiments, a CAR as described by the disclosure is humanized or fully human. In some embodiments, a CAR comprises one or more cytoplasmic domains that are capable of activating at least one of the normal effector functions of an immune cell in which the CAR is comprised in. In some embodiments, the cytoplasmic domain of the CAR comprises a CD3-zeta protein. In some embodiments, the arrangement of the elements of a CAR is the following exemplary, non-limiting arrangement:

[antigen binding domain]-[transmembrane domain]-[cytoplasmic domain]

In some embodiments, the antigen binding domain is an anti-B cell malignancy-associated antigen (e.g., BCMA). In some embodiments, the antigen binding domain is an anti-BCMA binding domain. BCMA is sometimes referred to as tumor necrosis factor receptor superfamily member 17 (TNFRSF17), a protein that in humans is encoded by the TNFRSF17 gene. In some embodiments, the antigen binding domain binds to the amino acid sequence set forth in SEQ ID NO: 1. An exemplary amino acid sequence for TNFRSF17 is provided below as SEQ ID NO: 1. In some embodiments, the antigen binding domain is an anti-CD19 binding domain. In some embodiments, the antigen binding domain is an anti-CS1 binding domain. In some embodiments, the antigen binding domain is an anti-CD38 binding domain. In some embodiments, the antigen binding domain is an anti-CD138 binding domain. In some embodiments, the antigen binding domain is an anti-CD30 binding domain. In some embodiments, the antigen binding domain is an anti-CD20 binding domain. In some embodiments, the antigen binding domain is an anti-CD25 binding domain.

TNFRSF17 (BCMA)-NP_001183.2 tumor necrosis factor
receptor superfamily member 17 [Homo sapiens]
(SEQ ID NO: 1)
MLQMAGQCSQNEYFDSLLHACIPCQLRCSSNTPPLTCQRYCNASVTNSVK
GTNAILWTCLGLSLIISLAVFVLMFLLRKINSEPLKDEFKNTGSGLLGMA
NIDLEKSRTGDEIILPRGLEYTVEECTCEDCIKSKPKVDSDHCFPLPAME
EGATILVTTKTNDYCKSLPAALSATEIEKSISAR

In some embodiments, the cytoplasmic domain is a domain capable of activating an effector function in an immune cell (e.g., an immunoreceptor tyrosine-based activation motif). In some embodiments, the cytoplasmic domain is an immunoreceptor tyrosine-based activation motif (ITAM). Examples of ITAM containing primary cytoplasmic signaling sequences that are of particular use in the invention include those derived from TCR zeta, FcR gamma, FcR beta, CD3 gamma, CD3 delta, CD3 epsilon, CD5, CD22, CD79a, CD79b, and CD66d. In some embodiments, the cytoplasmic signaling molecule of the CAR comprises a cytoplasmic signaling sequence derived from CD3zeta.

Between the extracellular domain (comprising the antigen binding domain) and the transmembrane domain of the CAR, or between the cytoplasmic domain and the transmembrane domain of the CAR, there may be incorporated a spacer or hinge domain. As used herein, the term “spacer domain” generally refers any oligo- or polypeptide that functions to link the transmembrane domain to the extracellular domain and/or the cytoplasmic domain in the polypeptide chain. As used herein, a hinge domain generally refers to any oligo- or polypeptide that functions to provide flexibility to the CAR, or domains thereof, and/or prevent steric hindrance of the CAR, or domains thereof. In some embodiments, a spacer or hinge domain may comprise up to 300 amino acids, preferably 10 to 100 amino acids and most preferably 5 to 20 amino acids. In some embodiments, the spacer or hinge domain comprises from 1 to 5, from 1 to 10, from 1 to 15, from 1 to 20, from 1 to 30, from 1 to 40, from 1 to 50, from 1 to 100, from 1 to 150, from 1 to 200, from 5 to 10, from 5 to 15, from 5 to 20, from 5 to 30, from 5 to 40, from 5 to 50, from 5 to 100, from 5 to 150, from 5 to 200, from 10 to 15, from 10 to 20, from 10 to 30, from 10 to 40, from 10 to 50, from 10 to 100, from 10 to 150, from 10 to 200, from 15 to 20, from 15 to 30, from 15 to 40, from 15 to 50, from 15 to 100, from 15 to 150, from 15 to 200, from 20 to 30, from 20 to 40, from 20 to 50, from 20 to 100, from 20 to 150, or from 20 to 200. It also should be appreciated that one or more spacer domains may be included in other regions of a CAR, as aspects of the disclosure are not limited in this respect.

It is to be understood that a CAR can include a region (e.g., an antigen binding domain, a transmembrane domain, a cytoplasmic domain, a signaling domain, and/or a linker, or any combination thereof) having a sequence provided herein or a variant thereof or a fragment of either one thereof (e.g., a variant and/or fragment that retains the function required for the CAR activity) can be included in a CAR protein as described herein. In some embodiments, a variant has 1, 2, 3, 4, 5, 6, 7, 8, 9, or 10 amino acid changes relative to the illustrated sequence. In some embodiments, a variant has a sequence that is at least 80%, at least 85%, at least 90%, at least 95%, at least 96%, at least 97%, at least 98%, at least 99%, or at least 99.5% identical to the illustrated sequence. In some embodiments, a fragment is 1-5, 5-10, 10-20, 20-30, 30-40, or 40-50 amino acids shorter than a sequence provided herein. In some embodiments, a fragment is shorter at the N-terminal, C-terminal, or both terminal regions of the sequence provided. In some embodiments, a fragment contains at least 80%, at least 85%, at least 90%, at least 95%, at least 96%, at least 97%, at least 98%, at least 99%, or at least 99.5% of the number of amino acids in a sequence described or provided herein (e.g., SEQ ID NOs: 4-6, and 8-12).

In some embodiments, any of the spacer and/or hinge sequences of the CAR comprise a G. In some embodiments, any of the spacer and/or hinge sequences of the CAR comprise a S. In some embodiments, any of the spacer and/or hinge sequences of the CAR are selected from one or more of the following exemplary sequences:

### Spacer Sequences:

(SEQ ID NO: 2)
GGGGS
(SEQ ID NO: 3)
GGGGSGGGGS
(SEQ ID NO: 13)
GGGGS x3
(SEQ ID NO: 14)
GSTSGGGSGGGSGGGGSS
(SEQ ID NO: 15)
GSTSGSGKPGSSEGSTKG
(SEQ ID NO: 16)
GGGGSGGG
(SEQ ID NO: 7)
GSTSGSGKPGSGEGSTKG

### Hinge Sequences:

(SEQ ID NO: 17)
VEPKSCDKTHTCPPCP
(SEQ ID NO: 18)
LDPKSSDKTHTCPPCP
(SEQ ID NO: 19)
VEPKSPDKTHTCPPCP
(SEQ ID NO: 20)
LDKTHTCPPCP

### Antigen Binding Domains

In some embodiments, the CAR of the invention comprises an antigen binding domain. The choice of binding domain depends upon the type and number of ligands that define the surface of a target cell. For example, the antigen binding domain may be chosen to recognize a ligand that acts as a cell surface marker on target cells associated with a particular disease state, such as cancer (e.g., multiple myeloma). Thus, examples of cell surface markers that may act as ligands for the antigen binding domain in the CAR of the invention include those associated with cancer cells and other forms of diseased cells, for example, autoimmune disease cells and pathogen infected cells. In some embodiments, the CAR of the invention is engineered to target a tumor antigen of interest by way of engineering a desired antigen binding domain that specifically binds to an antigen on a tumor cell. In the context of the present invention, “tumor antigen” refers to antigens that are common to specific hyperproliferative disorders such as cancer. The antigens discussed herein are merely included by way of example. The list is not intended to be exclusive and further examples will be readily apparent to those of skill in the art.

The antigen binding domain of the CAR may target, for example, BCMA. Other examples of target antigens include, but are not limited, to CD2, CD5, CD7, CD10, CD19, CD20, CD22, CD30, CD33, CD38, CD52, CD56, CD74, CD138, CD317, Her2, VEGFR2, EGFRviii, CXCR4, BCMA, GD2, GD3, and any other antigens over-expressed in target or diseased cells. Other antigens specific for cancer that may be targeted at taught in PCT publication No. WO2013/123061 (page 20), which is incorporated herein by reference with respect to the antigens recited therein.

The antigen binding domain can be any domain that binds to the antigen including but not limited to monoclonal antibodies, scFVs, polyclonal antibodies, synthetic antibodies, human antibodies, humanized antibodies, and antigen binding fragments thereof. In some instances, it is beneficial for the antigen binding domain to be derived from the same species in which the CAR will ultimately be used in. For example, for use in humans, it may be beneficial for the antigen binding domain of the CAR to comprise a human antibody, humanized antibody or antigen binding fragment thereof. Thus, in some embodiments, the antigen binding domain comprises a human antibody a humanized antibody or an antigen binding fragment thereof.

An antigen binding domain (e.g., an scFV) that “specifically binds” to a target or an epitope is a term understood in the art, and methods to determine such specific binding are also known in the art. A molecule is said to exhibit “specific binding” if it reacts or associates more frequently, more rapidly, with greater duration and/or with greater affinity with a particular target antigen than it does with alternative targets. An antibody “specifically binds” to a target antigen if it binds with greater affinity, avidity, more readily, and/or with greater duration than it binds to other substances. For example, an antigen binding domain (e.g., an scFV) that specifically binds to BCMA or an epitope therein is an antibody that binds this target antigen with greater affinity, avidity, more readily, and/or with greater duration than it binds to other antigens or other epitopes in the same antigen. It is also understood by reading this definition that, for example, an antigen binding domain (e.g., an scFV) that specifically binds to a first target antigen may or may not specifically bind to a second target antigen. As such, “specific binding” does not necessarily require (although it can include) exclusive binding. Generally, but not necessarily, reference to binding means specific binding. In some embodiments, antigen binding domains (e.g., scFVs) described herein have a suitable binding affinity to BCMA. As used herein, “binding affinity” refers to the apparent association constant or K_{A}. The K_{A} is the reciprocal of the dissociation constant (K_{D}). The antibody described herein may have a binding affinity (K_{A}) of at least 10⁵, 10⁶, 10⁷, 10⁸, 10⁹, 10¹⁰ M, or higher. An increased binding affinity corresponds to a decreased K_{D}. Higher affinity binding of an antibody to a first target relative to a second target can be indicated by a higher K_{A} (or a smaller numerical value K_{D}) for binding the first target than the K_{A} (or numerical value K_{D}) for binding the second target. In such cases, the antibody has specificity for the first target relative to the second target. Differences in binding affinity (e.g., for specificity or other comparisons) can be at least 1.5, 2, 3, 4, 5, 10, 15, 20, 37.5, 50, 70, 80, 91, 100, 500, 1000, 10,000 or 10⁵ fold.

Binding affinity can be determined by a variety of methods including equilibrium dialysis, equilibrium binding, gel filtration, ELISA, surface plasmon resonance, or spectroscopy (e.g., using a fluorescence assay). Exemplary conditions for evaluating binding affinity are in, e.g., TRIS-buffer (50 mM TRIS, 150 mM NaCl, 5 mM CaCl2 at pH7.5). These techniques can be used to measure the concentration of bound binding protein as a function of target protein concentration. The concentration of bound binding protein ([Bound]) is related to the concentration of free target protein ([Free]) and the concentration of binding sites for the binding protein on the target where (N) is the number of binding sites per target molecule by the following equation:

[Bound]=[N][Free]/(Kd+[Free])

It is not always necessary to make an exact determination of K_{A}, though, since sometimes it is sufficient to obtain a quantitative measurement of affinity, e.g., determined using a method such as ELISA or FACS analysis, is proportional to K_{A}, and thus can be used for comparisons, such as determining whether a higher affinity is, e.g., 2-fold higher, to obtain a qualitative measurement of affinity, or to obtain an inference of affinity, e.g., by activity in a functional assay, e.g., an in vitro or in vivo assay.

For in vivo use of antibodies in humans, it may be preferable to use human antibodies. Completely human antibodies are particularly desirable for therapeutic treatment of human subjects. Human antibodies can be made by a variety of methods known in the art including phage display methods using antibody libraries derived from human immunoglobulin sequences, including improvements to these techniques. See, also, U.S. Pat. Nos. 4,444,887 and 4,716,111; and PCT publications WO 98/46645, WO 98/50433, WO 98/24893, WO98/16654, WO 96/34096, WO 96/33735, and WO91/10741; each of which is incorporated herein by reference in its entirety. A human antibody can also be an antibody wherein the heavy and light chains are encoded by a nucleotide sequence derived from one or more sources of human DNA. Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. For example, it has been described that the homozygous deletion of the antibody heavy chain joining region (JH) gene in chimeric and germ-line mutant mice results in complete inhibition of endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, e.g., all or a portion of a polypeptide of the invention.

Antibodies directed against an antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies, including, but not limited to, IgG1 (gamma 1) and IgG3. For a detailed discussion of this technology for producing human antibodies and human monoclonal antibodies and protocols for producing such antibodies, see, e.g., PCT Publication Nos. WO2014/055771, WO 98/24893, WO 96/34096, and WO 96/33735; and U.S. Pat. Nos. 5,413,923; 5,625,126; 5,633,425; 5,569,825; 5,661,016; 5,545,806; 5,814,318; and 5,939,598, each of which is incorporated by reference herein in their entirety.

A “humanized” antibody retains a similar antigenic specificity as the original antibody, i.e., in the present invention, the ability to bind an antigen described herein, for example, BCMA.

In some embodiments, the antigen binding domain of the CAR of the invention targets BCMA. In some embodiments, the antigen binding moiety portion in the CAR of the invention is a humanized or fully human anti-BCMA scFV. In some embodiments, the anti-BCMA scFV comprises the scFV sequence of the CAR of any one of SEQ ID NOs: 4-6, and 8-12.

In some embodiments, the antigen binding domain of the CAR of the invention is specific for BCMA. In some embodiments, the antigen binding moiety portion in the CAR of the invention is an anti-BCMA scFV, such as a humanized or fully human anti-BCMA scFV. In some embodiments, the anti-BCMA scFV comprises the sequence(s) of the light and/or heavy chain variable regions with in the amino acid sequence of SEQ ID NOs: 4-6, and 8-12, or the complementarity determining regions (CDRs) contained within the light and/or heavy chain variable regions within the amino acid sequence of SEQ ID NOs 4-6, and 8-12. In some embodiments, the anti-BCMA scFV comprises the variable heavy chain (VH) and variable light chain (VL) sequences of any of the scFv sequences provided herein, or the complementarity determining regions (CDRs) contained within the scFv sequences provided herein.

### Transmembrane Domain

With respect to the transmembrane domain, the CAR can be designed to comprise a transmembrane domain that is fused to the extracellular domain (e.g., the antigen binding domain) of the CAR. Any transmembrane domain is contemplated for use herein as long as the domain is capable of anchoring a CAR comprising the domain to a cell membrane. In some embodiments, the transmembrane domain that naturally is associated with one of the domains in the CAR is used. In some instances, the transmembrane domain can be selected or modified by amino acid substitution to avoid binding of such domains to the transmembrane domains of the same or different surface membrane proteins to minimize interactions with other members of the receptor complex. One skilled in the art would appreciate that the full transmembrane domain, or portion thereof, is implemented with the cytoplasmic domain, or a portion thereof. Typically, the transmembrane and cytoplasmic domains used would be contiguous portions of the CD3-zeta protein sequence.

The transmembrane domain may be derived either from a natural or from a synthetic source. Where the source is natural, the domain may be derived from any membrane-bound or transmembrane protein. Transmembrane domains of particular use in this invention may be derived from (e.g., comprise at least the transmembrane domain(s) of) the alpha, beta or zeta chain of the T-cell receptor, CD28, CD3, CD45, CD4, CD5, CD8, CD9, CD16, CD22, CD33, CD37, CD64, CD80, CD86, CD134, CD137, and CD154. In some embodiments, the transmembrane domain is derived from a human CD8-alpha protein, a human CD28 protein, a human CD3-zeta protein, a human FcRγ protein, a CD27 protein, an OX40 protein, a human 4-1BB protein, or any modified version any of the foregoing that is capable of localizing in a cell membrane. In some embodiments, the transmembrane domain is derived from a human CD3-zeta protein. Transmembrane domains can be identified using any method known in the art or described herein, e.g., by using the UniProt Database.

In some embodiments, the transmembrane domain may be synthetic, in which case it will comprise predominantly hydrophobic residues such as leucine and valine. Preferably a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signaling domain of the CAR. A glycine-serine doublet provides a particularly suitable linker.

In some embodiments, the transmembrane domain in the CAR of the invention is the CD3-zeta transmembrane domain.

In some embodiments, the transmembrane domain in the CAR of the invention is a CD3-zeta transmembrane domain. An exemplary sequence of CD3-zeta is provided below, as well as an exemplary transmembrane domain sequence. In some embodiments, the CD3-zeta transmembrane domain comprises an exemplary transmembrane domain sequence provided herein, or a fragment or variant thereof that is capable of anchoring a CAR comprising the sequence to a cell membrane.

In some embodiments, the transmembrane domain in the CAR of the invention is a CD28 transmembrane domain. An exemplary sequence of CD28 is provided below, as well as an exemplary transmembrane domain sequence. In some embodiments, the CD28 transmembrane domain comprises the exemplary transmembrane domain sequence below, or a fragment or variant thereof that is capable of anchoring a CAR comprising the sequence to a cell membrane.

CD28 (amino acids 19-220)
(SEQ ID NO: 21)
NKILVKQSPMLVAYDNAVNLSCKYSYNLFSREFRASLHKGLDSAVEVCVV
YGNYSQQLQVYSKTGFNCDGKLGNESVTFYLQNLYVNQTDIYFCKIEVMY
PPPYLDNEKSNGTIIHVKGKHLCPSPLFPGPSKPFWVLVVVGGVLACYSL
LVTVAFIIFWVRSKRSRLLHSDYMNMTPRRPGPTRKHYQPYAPPRDFAAY
RS
CD28 (amino acids 153-179, transmembrane domain)
(SEQ ID NO: 22)
FWVLVVVGGVLACYSLLVTVAFIIFWV

In some embodiments, the CAR of the invention is comprises a region of CD28 that contains all or part of an extracellular domain, all or part of a transmembrane domain and all or part of a cytoplasmic domain. An exemplary sequence of a region of CD28 for inclusion in a CAR is provided below. In some embodiments, the CD28 transmembrane domain comprises the exemplary transmembrane domain sequence below, or a fragment or variant thereof that is capable of anchoring a CAR comprising the sequence to a cell membrane.

CD28 region
(SEQ ID NO: 23)
IEVMYPPPYLDNEKSNGTIIHVKGKHLCPSPLFPGPSKPFWVLVVVGGVL
ACYSLLVTVAFIIFWVRSKRSRLLHSDYMNMTPRRPGPTRKHYQPYAPPR
DFAAYRSAS

In some embodiments, the transmembrane domain of the CAR of the invention comprises a hinge domain such as a CD8 hinge domain. An exemplary CD8 hinge domain sequence is provided below. In some embodiments, the CD8 hinge domain comprises the exemplary sequence below, or a fragment or variant thereof that is capable of providing flexibility to or preventing steric hindrance of the CAR or the domain(s) attached to the hinge domain. In some instances, a variety of human hinges can be employed as well including the human Ig (immunoglobulin) hinge.

CD8 hinge domain
(SEQ ID NO: 24)
AKPTTTPAPRPPTPAPTIASQPLSLRPEACRPAAGGAVHTRGLDFACD

### Cytoplasmic Domain

In some embodiments, the cytoplasmic domain or otherwise the intracellular signaling domain of the CAR of the invention is responsible for activation of at least one of the normal effector functions of the immune cell in which the CAR has been placed in. The term “effector function” refers to a specialized function of a cell. Effector function of a T cell, for example, may be cytolytic activity or helper activity including the secretion of cytokines. Thus the term “intracellular signaling domain” refers to the portion of a protein which transduces the effector function signal and directs the cell to perform a specialized function. While usually the entire intracellular signaling domain can be employed, in many cases it is not necessary to use the entire domain. To the extent that a truncated portion of the intracellular signaling domain is used, such truncated portion may be used in place of the intact domain as long as it transduces the effector function signal. The term intracellular signaling domain is thus meant to include any truncated portion of the intracellular signaling domain sufficient to transduce the effector function signal.

In some embodiments, the cytoplasmic domain comprises a T cell activation domain that is capable of transducing a signal in a T cell (e.g., a cell proliferation or cytokine production signal). In some embodiments, the cytoplasmic domain comprises a human CD8-alpha protein, a human CD28 protein, a human CD3-zeta protein, a human FcRγ protein, a CD27 protein, an OX40 protein, a human 4-1BB protein, or modified version any of the foregoing. It should be appreciated that variants or fragments of any of the cytoplasmic domains that are capable of transducing a signal in a T cell are within the scope of this disclosure. In some embodiments, the T cell activation domain comprises a human CD3-zeta protein. In some embodiments, the T cell activation domain is a human CD3-zeta protein.

In some embodiments, the cytoplasmic domain comprises a CD3-zeta intracellular domain (e.g., CD3-zeta cytoplasmic domain). In some embodiments, the intracellular CD3-zeta cytoplasmic domain displays effector signaling function that enhances immune effector activities including, but not limited to cell proliferation and cytokine production. An exemplary CD3-zeta cytoplasmic domain sequence is provided herein, below. In some embodiments, the CD3-zeta cytoplasmic domain comprises the exemplary sequence below, or a fragment or variant thereof that, when included in a CAR, has the same or an improved function (such as cytolytic activity, cell proliferation or secretion of cytokines) compared to a CAR comprising the exemplary sequence below. The function may be tested using any suitable method known in the art.

In some embodiments, the cytoplasmic domain comprises a CD27 intracellular domain (e.g., CD27 cytoplasmic domain). In some embodiments, the intracellular CD27 cytoplasmic domain displays effector signaling function that enhances immune effector activities including, but not limited to cell proliferation and cytokine production. An exemplary CD27 cytoplasmic domain sequence is provided below. In some embodiments, the CD27 cytoplasmic domain comprises the exemplary sequence below, or a fragment or variant thereof that, when included in a CAR, has the same or an improved function (such as cytolytic activity, cell proliferation or secretion of cytokines) compared to a CAR comprising the exemplary sequence below. The function may be tested using any suitable method known in the art.

CD27 intracellular domain
(SEQ ID NO: 25)
QRRKYRSNKGESPVEPAEPCHYSCPREEEGSTIPIQEDYRKPEPACSP

Examples of other intracellular signaling domains for use in the CAR of the invention include the cytoplasmic sequences of the T cell receptor (TCR) and co-receptors that act in concert to initiate signal transduction following antigen receptor engagement, as well as any fragment or variant of these sequences and any synthetic sequence that has the same functional capability.

In some embodiments, signals generated through the endogenous TCR alone are insufficient for full activation of the T cell and that a secondary or co-stimulatory signal is also required. Thus, T cell activation can be mediated by two distinct classes of cytoplasmic signaling sequences: those that initiate antigen-dependent primary activation through the TCR (primary cytoplasmic signaling sequences) and those that act in an antigen-independent manner to provide a secondary or co-stimulatory signal (secondary cytoplasmic signaling sequences).

Primary cytoplasmic signaling sequences regulate primary activation of the TCR complex either in a stimulatory way, or in an inhibitory way. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences that are of particular use in the invention include those derived from TCR zeta, FcR gamma, FcR beta, CD3 gamma, CD3 delta, CD3 epsilon, CD5, CD22, CD79a, CD79b, and CD66d. It is particularly preferred that cytoplasmic signaling molecule in the CAR of the invention comprises a cytoplasmic signaling sequence derived from CD3-zeta. Exemplary CD3-zeta domain sequences are provided below. In some embodiments, the CD3-zeta signaling domain comprises one of the exemplary sequences below, or a fragment or variant thereof that, when included in a CAR, has the same or an improved function (such as cytolytic activity or secretion of cytokines) compared to a CAR comprising the exemplary sequence below.

CD3-zeta signaling domain
(SEQ ID NO: 26)
RVKFSRSADAPAYKQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR
CD3-zeta signaling domain
(SEQ ID NO: 27)
RVKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR

The cytoplasmic domain of the CAR can be designed to comprise a CD3-zeta signaling domain combined with any other desired cytoplasmic domain(s) useful in the context of the CAR of the invention. For example, the cytoplasmic domain of the CAR can comprise a CD3 zeta domain and a costimulatory signaling region. The costimulatory signaling region refers to a portion of the CAR comprising the intracellular domain of a costimulatory molecule. Exemplary co-stimulatory signaling regions include 4-1BB, CD21, CD28, CD27, CD127, ICOS, IL-15Rα, and OX40.

In some embodiments, the cytoplasmic domain of the CAR can be designed to comprise a CD27 cytoplasmic domain and a CD3-zeta signaling domain combined with any other desired cytoplasmic domain(s) useful in the context of the CAR of the disclosure. For example, the cytoplasmic domain of the CAR can comprise CD27 cytoplasmic domain, a CD3-zeta domain and a costimulatory signaling region. The costimulatory signaling region refers to a portion of the CAR comprising the intracellular domain of a costimulatory molecule. Example sequences of co-stimulatory signaling regions are shown below.

CD28 (amino acids 180-220, cytoplasmic domain)
(SEQ ID NO: 28)
RSKRSRLLHSDYMNMTPRRPGPTRKHYQPYAPPRDFAAYRS
4-1BB (CD137) intracellular TRAF binding domain
(SEQ ID NO: 29)
KRGRKKLLYIFKQPFMRPVQTTQEEDGCSCRFPEEEEGGCEL
ICOS intracellular domain
(SEQ ID NO: 30)
CWLTKKKYSSSVHDPNGEYMFMRAVNTAKKSRLTDVTL
OX40 intracellular domain
(SEQ ID NO: 31)
ALYLLRRDQRLPPDAHKPPGGGSFRTPIQEEQADAHSTLAKI
CD27 intracellular domain
(SEQ ID NO: 32)
QRRKYRSNKGESPVEPAEPCHYSCPREEEGSTIPIQEDYRKPEPACSP
CD127 intracellular domain
(SEQ ID NO: 33)
KRIKPIVWPSLPDHKKTLEHLCKKPRKNLNVSFNPESFLDCQIHRVDDIQ
ARDEVEGFLQDTFPQQLEESEKQRLGGDVQSPNCPSEDVVITPESFGRDS
SLTCLAGNVSACDAPILSSSRSLDCRESGKNGPHVYQDLLLSLGTTNSTL
PPPFSLQSGILTLNPVAQGQPILTSLGSNQEEAYVTMSSFYQNQ

The cytoplasmic signaling sequences within the cytoplasmic signaling portion of the CAR of the invention may be linked to each other in a random or specified order. Optionally, a short oligo- or polypeptide linker or spacer, preferably between 5 and 20 amino acids in length may be inserted between cytoplasmic domains. In some embodiments, the cytoplasmic signaling sequences are linked via any of the spacer or hinge domains provided herein. In some embodiments, the cytoplasmic signaling sequences are linked via a GGGGS (SEQ ID NO: 2), GGGGSGGGGS (SEQ ID NO: 3), or GGGGSGGGGSGGGGS (SEQ ID NO: 13).

In some embodiments, a CAR comprises or consists of the any one of SEQ ID NOs: 4, 5, 6, 8, 9, 10, 11, or 12. In some embodiments, the above exemplary, non-limiting arrangements are from left to right, N-terminus to C-terminus of the CAR. The CAR may comprise or further comprise any other combination of elements as described herein.

### Vectors

In some embodiments, the present invention encompasses a DNA construct comprising sequences encoding a CAR, wherein the sequence comprises the nucleic acid sequence of an antigen binding domain operably linked to the nucleic acid sequence of transmembrane domain and a cytoplasmic domain. An exemplary cytoplasmic domain that can be used in a CAR of the invention includes but is not limited to the signaling domain of CD3-zeta. In some embodiments, a CAR comprises the intracellular domain of CD28, 4-1BB, and/or CD27 and the signaling domain of CD3-zeta.

In some embodiments, any of the above exemplary, non-limiting arrangements are from left to right, N-terminus to C-terminus of the CAR. The CAR may comprise or further comprise any other combination of elements as described herein.

The nucleic acid sequences coding for the desired molecules (e.g., any of the CARs provided herein) can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

The present invention also provides vectors in which a DNA of the present invention is inserted. Vectors derived from retroviruses such as the lentivirus are suitable tools to achieve long-term gene transfer since they allow long-term, stable integration of a transgene and its propagation in daughter cells. Lenti viral vectors have the added advantage over vectors derived from onco-retroviruses such as murine leukemia viruses in that they can transduce non-proliferating cells, such as hepatocytes. They also have the added advantage of low immunogenicity. In another embodiment, the desired CAR can be expressed in the cells by way of transposons.

In brief summary, the expression of natural or synthetic nucleic acids encoding CARs is typically achieved by operably linking a nucleic acid encoding the CAR polypeptide or portions thereof to a promoter, and incorporating the construct into an expression vector. The vectors can be suitable for replication and integration into eukaryotes. Typical cloning vectors contain transcription and translation terminators, initiation sequences, and promoters useful for regulation of the expression of the desired nucleic acid sequence. The expression constructs of the present invention may also be used for nucleic acid immunization and gene therapy, using standard gene delivery protocols. Methods for gene delivery are known in the art. See, e.g., U.S. Pat. Nos. 5,399,346, 5,580,859, 5,589,466, incorporated by reference herein in their entireties. In another embodiment, the invention provides a gene therapy vector.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, retrovirus vectors are used. A number of retrovirus vectors are known in the art. In some embodiments, lentivirus vectors are used.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Factor-1a (EF-1a). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter. In some embodiments, the promoter is a EF-1a promoter.

In order to assess the expression of a CAR polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like, and fluorescent genes such as GFP, YFP, RFP and the like. In some embodiments, reporter genes or selectable marker genes are excluded from a CAR polypeptide used in a therapy as described herein.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity, antibiotic resistance or fluorescence. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means. In some embodiments, the host cell is a T cell.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York). A preferred method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the inhibitor of the present invention, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

### RNA Transfection

In some embodiments, the genetically modified T cells of the invention are modified through the introduction of RNA (e.g., an mRNA comprises a sequence encoding a CAR as described herein). In some embodiments, the RNA (e.g., mRNA) encodes a CAR comprising an antigen recognition moiety, a transmembrane domain, and a T cell activation moiety. In some embodiments, the RNA (e.g., mRNA) encodes a CAR that does not occur in nature. In some embodiments, the RNA (e.g., mRNA) encodes any of the CARs provided herein. In some embodiments, an in vitro transcribed RNA CAR can be introduced to a cell as a form of transient transfection. The RNA is produced by in vitro transcription using a polymerase chain reaction (PCR)-generated template. DNA of interest from any source can be directly converted by PCR into a template for in vitro mRNA synthesis using appropriate primers and RNA polymerase. The source of the DNA can be, for example, genomic DNA, plasmid DNA, phage DNA, cDNA, synthetic DNA sequence or any other appropriate source of DNA. The desired template for in vitro transcription is the CAR of the present invention. For example, in some embodiments, the template for the RNA CAR comprises an extracellular domain comprising an anti-BCMA scFv; a transmembrane domain; and a cytoplasmic domain comprises the signaling domain of CD3-zeta.

RNA can be introduced into target cells using any of a number of different methods, for instance, commercially available methods which include, but are not limited to, electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendort, Hamburg Germany), cationic liposome mediated transfection using lipofection, polymer encapsulation, peptide mediated transfection, or biolistic particle delivery systems such as “gene guns” (see, for example, Nishikawa, et al. Hum Gene Ther., 12(8):861-70 (2001). In some embodiments, RNA can be transduced into target cells using particle-based methods. Exemplary particle-based methods include, without limitation, precious-metal-based, liposomal, polymer-based, or endosome-based nanoparticles. Polymer-based nanoparticles may include, for example, poly[beta]-amino esters or other chemicals with biodegradable and pH-sensitive properties. Nanoparticles may be coated with, for example, polyglutamic acid (PGA) and/or antibodies or antibody-fragments targeting cell-membrane antigens, for example CD4, CD8, CD3, CD56, to facilitiate uptake.

### Genetically Modified Immune Cells

In some embodiments, nucleic acid sequences (e.g., RNA or DNA) encoding a CAR (e.g., any of the CARs as described herein) are delivered into cells (e.g., T cells or NK cells). In some embodiments, an RNA (e.g., mRNA) encoding a CAR (e.g., any CAR provided herein) is introduced into a cell, for example via electroporation. In some embodiments, a nucleic acid encoding a CAR is introduced into a cell using a retroviral or lentiviral vector.

CAR-expressing retroviral and lentiviral vectors can be delivered into different types of eukaryotic cells as well as into tissues and whole organisms using transduced cells as carriers or cell-free local or systemic delivery of encapsulated, bound or naked vectors. The method used can be for any purpose where stable expression is required or sufficient.

In another embodiment, the desired CAR can be expressed in the cells (e.g., T cells or NK cells) by way of transposons.

The disclosed methods can be applied to the modulation of immune cell (e.g., T cell or NK cell) activity in basic research and therapy, in the fields of cancer, stem cells, acute and chronic infections, and autoimmune diseases, including the assessment of the ability of the genetically modified T cell or NK cell to kill a target cell, e.g., a target cancer cell.

The methods also provide the ability to control the level of expression over a wide range by changing, for example, the amount of RNA (e.g., mRNA) delivered to the cells, modifications to RNA that modulate (e.g., increase or decrease) the half-life of the RNA, (e.g., synthetic nucleotides, poly-A tails, and/or cap structures). In some embodiments, the level of expression is controlled by changing the promoter or the amount of input vector, making it possible to individually regulate the expression level. For example, varying of different intracellular effector/costimulator domains on multiple chimeric receptors in the same cell allows determination of the structure of the receptor combinations which assess the highest level of cytotoxicity against multi-antigenic targets, and at the same time lowest cytotoxicity toward normal cells.

In some embodiments, any of the RNAs (e.g., mRNAs) provided herein, may comprise one or more stabilizing elements. Naturally-occurring eukaryotic mRNA molecules have been found to contain stabilizing elements, including, but not limited to untranslated regions (UTR) at their 5′-end (5′UTR) and/or at their 3′-end (3′UTR), in addition to other structural features, such as a 5′-cap structure or a 3′-poly(A) tail. Both the 5′UTR and the 3′UTR are typically transcribed from the genomic DNA and are elements of the premature mRNA. Characteristic structural features of mature mRNA, such as the 5′-cap and the 3′-poly(A) tail are usually added to the transcribed (premature) mRNA during mRNA processing. The 3′-poly(A) tail is typically a stretch of adenine nucleotides added to the 3′-end of the transcribed mRNA. It can comprise up to about 400 adenine nucleotides. In some embodiments the length of the 3′-poly(A) tail may be an essential element with respect to the stability of the individual mRNA.

Stabilizing elements may include for instance a histone stem-loop. A stem-loop binding protein (SLBP), a 32 kDa protein has been identified. It is associated with the histone stem-loop at the 3′-end of the histone messages in both the nucleus and the cytoplasm. Its expression level is regulated by the cell cycle; it is peaks during the S-phase, when histone mRNA levels are also elevated. The protein has been shown to be essential for efficient 3′-end processing of histone pre-mRNA by the U7 snRNP. SLBP continues to be associated with the stem-loop after processing, and then stimulates the translation of mature histone mRNAs into histone proteins in the cytoplasm. The RNA binding domain of SLBP is conserved through metazoa and protozoa; its binding to the histone stem-loop depends on the structure of the loop. The minimum binding site includes at least three nucleotides 5′ and two nucleotides 3′ relative to the stem-loop.

In some embodiments, the RNA includes a coding region, at least one histone stem-loop, and optionally, a poly(A) sequence or polyadenylation signal. The poly(A) sequence or polyadenylation signal generally should enhance the expression level of the encoded protein. The encoded protein, in some embodiments, is not a histone protein, a reporter protein (e.g. Luciferase, GFP, EGFP, β-Galactosidase, EGFP), or a marker or selection protein (e.g. alpha-Globin, Galactokinase and Xanthine:guanine phosphoribosyl transferase (GPT)).

In some embodiments, the combination of a poly(A) sequence or polyadenylation signal and at least one histone stem-loop, even though both represent alternative mechanisms in nature, acts synergistically to increase the protein expression beyond the level observed with either of the individual elements. It has been found that the synergistic effect of the combination of poly(A) and at least one histone stem-loop does not depend on the order of the elements or the length of the poly(A) sequence.

The disclosure further provides other technologies used to modify immune cells. For example, the methods include, without limitation, use of site-specific nucleases, for example ZFNs (zinc-finger nucleases), TALENs (transcription activator-like effector nucleases), CRISPR/Cas9, RFNs (dimeric CRISPR RNA-guided Fokl nucleases), and eMAGE (eukaryotic multiplex automated genome engineering) to modify nucleic acids of immune cells.

### Sources of Immune Cells

Prior to expansion and genetic modification of the immune cells (e.g., T cells) of the invention, a source of immune cells (e.g., T cells) is obtained from a subject. Immune cells (e.g., T cells) can be obtained from a number of sources, including peripheral blood mononuclear cells, bone marrow, lymph node tissue, cord blood, thymus tissue, tissue from a site of infection, ascites, pleural effusion, spleen tissue, and tumors. The immune cells (e.g., T cells) may also be generated from induced pluripotent stem cells or hematopoietic stem cells or progenitor cells. In some embodiments of the present invention, any number of immune cell lines, including but not limited to T cell and NK cell lines, available in the art, may be used. In some embodiments of the present invention, immune cells (e.g., T cells) can be obtained from a unit of blood collected from a subject using any number of techniques known to the skilled artisan, such as Ficoll™ separation. In some embodiments, cells from the circulating blood of an individual are obtained by apheresis. The apheresis product typically contains lymphocytes, including T cells, monocytes, granulocytes, B cells, NK cells, other nucleated white blood cells, red blood cells, and platelets. In some embodiments, the cells collected by apheresis may be washed to remove the plasma fraction and to place the cells in an appropriate buffer or media for subsequent processing steps. In some embodiments of the invention, the cells are washed with phosphate buffered saline (PBS). In an alternative embodiment, the wash solution lacks calcium and may lack magnesium or may lack many if not all divalent cations. Again, surprisingly, initial activation steps in the absence of calcium lead to magnified activation. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca²⁺-free, Mg²⁺-free PBS, PlasmaLyte A, or other saline solution with or without buffer. Alternatively, the undesirable components of the apheresis sample may be removed and the cells directly resuspended in culture media.

In another embodiment, immune cells (e.g., T cells) are isolated from peripheral blood lymphocytes by lysing the red blood cells and depleting the monocytes, for example, by centrifugation through a PERCOLL™ gradient or by counterflow centrifugal elutriation. A specific subpopulation of T cells, such as CD8⁺, CD3⁺, CD28⁺, CD4⁺, CD45RA⁺, and CD45RO⁺ T cells, can be further isolated by positive or negative selection techniques. For example, in some embodiments, T cells are isolated by incubation with anti-CD3/anti-CD28 (i.e., 3×28)-conjugated beads, such as DYNABEADS® M-450 CD3/CD28 T, for a time period sufficient for positive selection of the desired T cells. In some embodiments, the time period is about 30 minutes. In a further embodiment, the time period ranges from 30 minutes to 36 hours or longer and all integer values there between. In a further embodiment, the time period is at least 1, 2, 3, 4, 5, or 6 hours. In yet another preferred embodiment, the time period is 10 to 24 hours. In one preferred embodiment, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types, such in isolating tumor infiltrating lymphocytes (TIL) from tumor tissue or from immune-compromised individuals. Further, use of longer incubation times can increase the efficiency of capture of CD8+ T cells. Thus, by simply shortening or lengthening the time T cells are allowed to bind to the CD3/CD28 beads and/or by increasing or decreasing the ratio of beads to T cells (as described further herein), subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. The skilled artisan would recognize that multiple rounds of selection can also be used in the context of this invention. In certain embodiments, it may be desirable to perform the selection procedure and use the “unselected” cells in the activation and expansion process. “Unselected” cells can also be subjected to further rounds of selection.

Enrichment of a T cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD8⁺ cells by negative selection, a monoclonal antibody cocktail typically includes antibodies to CD14, CD20, CD11b, CD16, HLA-DR, and CD4. In certain embodiments, it may be desirable to enrich for or positively select for regulatory T cells which typically express CD4⁺, CD25⁺, CD62L^{h1}, GITR⁺, and FoxP3⁺.

Alternatively, in certain embodiments, T regulatory cells are depleted by anti-C25 conjugated beads or other similar method of selection.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in some embodiments, a concentration of 2 billion cells/ml is used. In some embodiments, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (e.g., leukemic blood, tumor tissue, etc.). Such populations of cells may have therapeutic value and would be desirable to obtain. For example, using high concentration of cells allows more efficient selection of CD8⁺ T cells that normally have weaker CD28 expression.

In a related embodiment, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4⁺ T cells express higher levels of CD28 and are more efficiently captured than CD8⁺ T cells in dilute concentrations. In some embodiments, the concentration of cells used is 5×10⁶/ml. In other embodiments, the concentration used can be from about 1×10⁵/ml to 1×10⁶/ml, and any integer value in between.

In other embodiments, the cells may be incubated on a rotator for varying lengths of time at varying speeds at either 2-10° C. or at room temperature.

T cells for stimulation can also be frozen after a washing step. Wishing not to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen. In certain embodiments, cryopreserved cells are thawed and washed as described herein and allowed to rest for one hour at room temperature prior to activation using the methods of the present invention.

Also contemplated in the context of the invention is the collection of blood samples or apheresis product from a subject at a time period prior to when the expanded cells as described herein might be needed. As such, the source of the cells to be expanded can be collected at any time point necessary, and desired cells, such as T cells, isolated and frozen for later use in T cell therapy for any number of diseases or conditions that would benefit from T cell therapy, such as those described herein. In some embodiments a blood sample or an apheresis is taken from a generally healthy subject. In certain embodiments, a blood sample or an apheresis is taken from a generally healthy subject who is at risk of developing a disease, but who has not yet developed a disease, and the cells of interest are isolated and frozen for later use. In certain embodiments, the T cells may be expanded, frozen, and used at a later time. In certain embodiments, samples are collected from a patient shortly after diagnosis of a particular disease as described herein but prior to any treatments. In a further embodiment, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and FK506, antibodies, or other immunoablative agents such as CAMPATH, anti-CD3 antibodies, Cytoxan, fludarabine, cyclosporin, FK506, rapamycin, mycophenolic acid, steroids, FR901228, and irradiation. These drugs inhibit either the calcium dependent phosphatase calcineurin (cyclosporine and FK506) or inhibit the p70S6 kinase that is important for growth factor induced signaling (rapamycin) (Liu et al., Cell 66:807-815, 1991; Henderson et al., Immun. 73:316-321, 1991; Bierer et al., Curr. Opin. Immun. 5:763-773, 1993). In a further embodiment, the cells are isolated for a patient and frozen for later use in conjunction with (e.g., before, simultaneously or following) bone marrow or stem cell transplantation, T cell ablative therapy using either chemotherapy agents such as, fludarabine, external-beam radiation therapy (XRT), cyclophosphamide, or antibodies such as OKT3 or CAMPATH. In another embodiment, the cells are isolated prior to and can be frozen for later use for treatment following B-cell ablative therapy such as agents that react with CD20, e.g., Rituxan.

In a further embodiment of the present invention, T cells are obtained from a patient directly following treatment. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present invention to collect blood cells, including T cells, dendritic cells, or other cells of the hematopoietic lineage, during this recovery phase. Further, in certain embodiments, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

### Activation and Expansion of T Cells

Whether prior to or after genetic modification of the T cells to express a desirable CAR (e.g., any of the CARs provided herein), the T cells can be activated and expanded generally using methods as described, for example, in U.S. Pat. Nos. 6,352,694 and 6,534,055.

Generally, T cells of the invention are expanded by contact with a surface having attached thereto an agent that stimulates a CD3/TCR complex associated signal and a ligand that stimulates a co-stimulatory molecule on the surface of the T cells. In particular, T cell populations may be stimulated as described herein, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4⁺ T cells or CD8⁺ T cells, an anti-CD3 antibody and an anti-CD28 antibody. Examples of an anti-CD28 antibody include 9.3, B-T3, XR-CD28 (Diaclone, Besancon, France) can be used as can other methods commonly known in the art (Berg et al., Transplant Proc. 30(8):3975-3977, 1998; Haanen et al., J. Exp. Med. 190(9): 13191328, 1999; Garland et al., J. Immunol Meth. 227(1-2):53-63, 1999).

In certain embodiments, the primary stimulatory signal and the co-stimulatory signal for the T cell may be provided by different protocols. For example, the agents providing each signal may be in solution or coupled to a surface. When coupled to a surface, the agents may be coupled to the same surface (i.e., in “cis” formation) or to separate surfaces (i.e., in “trans” formation). Alternatively, one agent may be coupled to a surface and the other agent in solution. In some embodiments, the agent providing the co-stimulatory signal is bound to a cell surface and the agent providing the primary activation signal is in solution or coupled to a surface. In certain embodiments, both agents can be in solution. In another embodiment, the agents may be in soluble form, and then cross-linked to a surface, such as a cell expressing Fc receptors or an antibody or other binding agent which will bind to the agents. In this regard, see for example, U.S. Patent Application Publication Nos. 20040101519 and 20060034810 for artificial antigen presenting cells (aAPCs) that are contemplated for use in activating and expanding T cells in the present invention.

In some embodiments, the two agents are immobilized on beads, either on the same bead, i.e., “cis,” or to separate beads, i.e., “trans.” By way of example, the agent providing the primary activation signal is an anti-CD3 antibody or an antigen-binding fragment thereof and the agent providing the co-stimulatory signal is an anti-CD28 antibody or antigen-binding fragment thereof; and both agents are co-immobilized to the same bead in equivalent molecular amounts. In some embodiments, a 1:1 ratio of each antibody bound to the beads for CD4⁺ T cell expansion and T cell growth is used. In certain aspects of the present invention, a ratio of anti CD3:CD28 antibodies bound to the beads is used such that an increase in T cell expansion is observed as compared to the expansion observed using a ratio of 1:1. In one particular embodiment an increase of from about 1 to about 3 fold is observed as compared to the expansion observed using a ratio of 1:1. In some embodiments, the ratio of CD3:CD28 antibody bound to the beads ranges from 100:1 to 1:100 and all integer values there between. In one aspect of the present invention, more anti-CD28 antibody is bound to the particles than anti-CD3 antibody, i.e., the ratio of CD3:CD28 is less than one. In certain embodiments of the invention, the ratio of anti CD28 antibody to anti CD3 antibody bound to the beads is greater than 2:1. In one particular embodiment, a 1:100 CD3:CD28 ratio of antibody bound to beads is used. In another embodiment, a 1:75 CD3:CD28 ratio of antibody bound to beads is used. In a further embodiment, a 1:50 CD3:CD28 ratio of antibody bound to beads is used. In another embodiment, a 1:30 CD3:CD28 ratio of antibody bound to beads is used. In one preferred embodiment, a 1:10 CD3:CD28 ratio of antibody bound to beads is used. In another embodiment, a 1:3 CD3:CD28 ratio of antibody bound to the beads is used. In yet another embodiment, a 3:1 CD3:CD28 ratio of antibody bound to the beads is used.

Ratios of particles to cells from 1:500 to 500:1 and any integer values in between may be used to stimulate T cells or other target cells. As those of ordinary skill in the art can readily appreciate, the ratio of particles to cells may depend on particle size relative to the target cell. For example, small sized beads could only bind a few cells, while larger beads could bind many. In certain embodiments the ratio of cells to particles ranges from 1:100 to 100:1 and any integer values in-between and in further embodiments the ratio comprises 1:9 to 9:1 and any integer values in between, can also be used to stimulate T cells. The ratio of anti-CD3- and anti-CD28-coupled particles to T cells that result in T cell stimulation can vary as noted above, however certain preferred values include 1:100, 1:50, 1:40, 1:30, 1:20, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, and 15:1 with one preferred ratio being at least 1:1 particles per T cell. In some embodiments, a ratio of particles to cells of 1:1 or less is used. In one particular embodiment, a preferred particle:cell ratio is 1:5. In further embodiments, the ratio of particles to cells can be varied depending on the day of stimulation. For example, in some embodiments, the ratio of particles to cells is from 1:1 to 10:1 on the first day and additional particles are added to the cells every day or every other day thereafter for up to 10 days, at final ratios of from 1:1 to 1:10 (based on cell counts on the day of addition). In one particular embodiment, the ratio of particles to cells is 1:1 on the first day of stimulation and adjusted to 1:5 on the third and fifth days of stimulation. In another embodiment, particles are added on a daily or every other day basis to a final ratio of 1:1 on the first day, and 1:5 on the third and fifth days of stimulation. In another embodiment, the ratio of particles to cells is 2:1 on the first day of stimulation and adjusted to 1:10 on the third and fifth days of stimulation. In another embodiment, particles are added on a daily or every other day basis to a final ratio of 1:1 on the first day, and 1:10 on the third and fifth days of stimulation. One of skill in the art will appreciate that a variety of other ratios may be suitable for use in the present invention. In particular, ratios will vary depending on particle size and on cell size and type.

In further embodiments of the present invention, the cells, such as T cells, are combined with agent-coated beads, the beads and the cells are subsequently separated, and then the cells are cultured. In an alternative embodiment, prior to culture, the agent-coated beads and cells are not separated but are cultured together. In a further embodiment, the beads and cells are first concentrated by application of a force, such as a magnetic force, resulting in increased ligation of cell surface markers, thereby inducing cell stimulation.

By way of example, cell surface proteins may be ligated by allowing paramagnetic beads to which anti-CD3 and anti-CD28 are attached (3×28 beads) to contact the T cells. In some embodiments the cells (for example, 10⁴ to 10⁹ T cells) and beads (for example, DYNABEADS® M-450 CD3/CD28 T paramagnetic beads at a ratio of 1:1) are combined in a buffer, preferably PBS (without divalent cations such as, calcium and magnesium). Again, those of ordinary skill in the art can readily appreciate any cell concentration may be used. For example, the target cell may be very rare in the sample and comprise only 0.01% of the sample or the entire sample (i.e., 100%) may comprise the target cell of interest. Accordingly, any cell number is within the context of the present invention. In certain embodiments, it may be desirable to significantly decrease the volume in which particles and cells are mixed together (i.e., increase the concentration of cells), to ensure maximum contact of cells and particles. For example, in some embodiments, a concentration of about 2 billion cells/ml is used. In another embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells. Such populations of cells may have therapeutic value and would be desirable to obtain in certain embodiments. For example, using high concentration of cells allows more efficient selection of CD8+ T cells that normally have weaker CD28 expression.

In some embodiments of the present invention, the mixture may be cultured for several hours (about 3 hours) to about 14 days or any hourly integer value in between. In another embodiment, the mixture may be cultured for 21 days. In some embodiments of the invention the beads and the T cells are cultured together for about eight days. In another embodiment, the beads and T cells are cultured together for 2-3 days. Several cycles of stimulation may also be desired such that culture time of T cells can be 60 days or more. Conditions appropriate for T cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-γ, IL-4, IL-7, GM-CSF, IL-10, IL-12, IL-15, TGFp, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, a-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂).

T cells that have been exposed to varied stimulation times may exhibit different characteristics. For example, typical blood or apheresed peripheral blood mononuclear cell products have a helper T cell population (¾, CD4⁺) that is greater than the cytotoxic or suppressor T cell population (T_{c}, CD8⁺). Ex vivo expansion of T cells by stimulating CD3 and CD28 receptors produces a population of T cells that prior to about days 8-9 consists predominately of ¾ cells, while after about days 8-9, the population of T cells comprises an increasingly greater population of Tc cells. Accordingly, depending on the purpose of treatment, infusing a subject with a T cell population comprising predominately of T_{H} cells may be advantageous. Similarly, if an antigen-specific subset of Tc cells has been isolated it may be beneficial to expand this subset to a greater degree.

Further, in addition to CD4 and CD 8 markers, other phenotypic markers vary significantly, but in large part, reproducibly during the course of the cell expansion process. Thus, such reproducibility enables the ability to tailor an activated T cell product for specific purposes.

### Therapeutic Application

Some aspects of the disclosure provide methods for treating a B cell malignancy. In some embodiments, the disclosure provides a method for treating a B cell malignancy in an individual, the method comprising administering to the individual a cell therapy according to the present invention.

In some embodiments, the B cell malignancy is selected from the group consisting of multiple myeloma, plasmacytoma, Hodgkin lymphoma, mantle cell lymphoma, hairy cell leukemia, Burkitt's lymphoma, MALT lymphoma, chronic lymphocytic leukemia, and acute lymphoblastic leukemia. In some embodiments, the B cell malignancy is multiple myeloma. It should be appreciated that the methods provided herein may include the treatment of additional B cell malignancies and the list of B cell malignancies provided herein is not meant to be limiting.

In some embodiments, the method is a first-line therapy. First line therapy refers to the treatment regimen or regimens that are generally accepted by the medical establishment for initial treatment for a given type and stage of cancer. In some embodiments, the method is a second-line therapy. Second line therapies are those tried when the first one(s) do not work adequately.

In some embodiments, the individual has not undergone a bone marrow transplant. In some embodiments, the individual has not undergone chemotherapy. In some embodiments, the individual has undergone a bone marrow transplant. In some embodiments, the individual has undergone chemotherapy

The method can use any of the compositions (e.g., cell therapies) provided herein. For example the method can use any of the cell therapies described under “Cell Therapy Product,” supra.

Some aspects of the disclosure provide methods for treating B cell-associated diseases other than B cell malignancies. In some embodiments, the disclosure provides a method for treating a B cell-associated disease in an individual, the method comprising administering to the individual a cell therapy according to the present invention. In some embodiments, the B cell-associated disease is selected from the group consisting of systemic lupus erythematosus (SLE), rheumatoid arthritis, psoriasis, inflammatory bowel disease, celiac sprue, pernicious anemia, sceleroderma, Graves disease, Sjögren syndrome, autoimmune hemolytic anemia (AIHA), myasthenia gravis, cryoglobulinemia, thrombotic thrombocytopenic purpura (TTP), allograft rejection (e.g., transplant rejection of lung, kidney, heart, intestine, liver, pancreas, etc.), pemphigus vulgaris, vitiligo, Hashimoto's disease, Addison's disease, reactive arthritis, and type 1 diabetes.

The method can use any of the compositions (e.g., cell therapies) provided herein. For example the method can use any of the cell therapies described under “Cell Therapy Product,” supra.

In some embodiments, the present invention provides a cell (e.g., T cell) modified to express a CAR (e.g., any CAR provided herein) that comprises an antigen binding domain (e.g., that binds BCMA), a transmembrane domain, and a cytoplasmic domain (e.g., CD3-zeta and/or any other cytoplasmic domains described herein). In some embodiments, a cell is modified to express a CAR comprising an antigen binding domain, a transmembrane domain, and a cytoplasmic domain having a CD3-zeta domain and/or any other cytoplasmic domains provided herein. In some embodiments, a cell is modified to express a CAR comprising an antigen binding domain (e.g., a scFV specific for BCMA), a transmembrane domain, and a cytoplasmic domain. Therefore, in some instances, the transduced T cell can elicit a CAR-mediated T-cell response.

In some embodiments, the invention provides the use of a CAR to redirect the specificity of a primary T cell to an antigen, such as a tumor antigen (e.g., BCMA). Thus, in some embodiments, the present invention also provides a method for stimulating a T cell-mediated immune response to a target cell population or tissue in a mammal comprising the step of administering to the mammal a T cell that expresses a CAR, wherein the CAR comprises an antigen binding domain (e.g., BCMA scFV), a transmembrane domain, and a cytoplasmic domain comprising a CD3-zeta and/or any other cytoplasmic domains described herein.

In some embodiments, a method for stimulating a T cell-mediated immune response to a target cell population or tissue in a mammal comprising the step of administering to the mammal a T cell that expresses a CAR, wherein the CAR comprises an antigen binding domain (e.g., BCMA scFV), a transmembrane domain, and a cytoplasmic domain having a CD3-zeta and/or any other cytoplasmic domains described herein.

In some embodiments, the disclosure provides a method for stimulating a T cell-mediated immune response to a target cell population or tissue in a mammal comprising the step of administering to the mammal a T cell that expresses a CAR, wherein the CAR comprises an antigen binding domain (e.g., BCMA scFV), a transmembrane domain, and a cytoplasmic domain (e.g., comprising CD3-zeta and/or any other cytoplasmic domains described herein).

In some embodiments, the present invention includes a type of cellular therapy where T cells are genetically modified to express a CAR and the CAR T cell is infused to a recipient in need thereof. The infused cell is able to kill cells expressing the antigen, e.g., tumor cells, in the recipient. Unlike antibody therapies, CAR T cells are able to replicate in vivo resulting in long-term persistence that can lead to sustained tumor control.

In some embodiments, the CAR T cells of the invention can undergo robust in vivo T cell expansion and can persist for an extended amount of time.

While the data disclosed herein disclose RNA encoding a CDR having an anti-BCMA scFv, a transmembrane domain, and a CD3-zeta signaling domain, the invention should be construed to include any number of variations for each of the components of the construct as described elsewhere herein. That is, the invention includes the use of any antigen binding domain in the CAR to generate a CAR-mediated T-cell response specific to the antigen binding domain. For example, the antigen binding domain in the CAR of the invention can target a tumor antigen for the purposes of treat cancer (e.g., multiple myeloma).

In some embodiments, the antigen binding domain portion of the CAR of the invention is designed to treat a particular cancer. For example, the antigen binding domain portion of the CAR of the invention is designed to treat a B cell malignancy, such as multiple myeloma, plasmacytoma, Hodgkin lymphoma, mantle cell lymphoma, hairy cell leukemia, Burkitt's lymphoma, MALT lymphoma, chronic lymphocytic leukemia, or acute lymphoblastic leukemia. In some embodiments, the antigen binding domain portion of the CAR of the invention is designed to treat a B cell-associated disease, such as systemic lupus erythematosus (SLE), rheumatoid arthritis, psoriasis, inflammatory bowel disease, celiac sprue, pernicious anemia, sceleroderma, Graves disease, Sjögren syndrome, or type 1 diabetes. As another example, the CAR may be designed to target BCMA for treating B cell malignancies, or CD30 for treating Hodgkin's lymphoma or certain T cell lymphoma, or GD2 for treating small cell neuroendocrine cancer or small cell lung cancer, and neuronal cancer.

The CAR-modified T cells of the invention may also serve as a type of vaccine for ex vivo immunization and/or in vivo therapy in a mammal. Preferably, the mammal is a human.

With respect to ex vivo immunization, at least one of the following occurs in vitro prior to administering the cell into a mammal: i) expansion of the cells, ii) introducing a nucleic acid encoding a CAR to the cells, and/or iii) cryopreservation of the cells.

Ex vivo procedures are well known in the art and are discussed more fully below. Briefly, cells are isolated from a mammal (preferably a human) and genetically modified (e.g., transduced or transfected in vitro) with a vector expressing a CAR disclosed herein. The CAR-modified cell can be administered to a mammalian recipient to provide a therapeutic benefit. The mammalian recipient may be a human and the CAR-modified cell can be autologous with respect to the recipient. Alternatively, the cells can be allogeneic, syngeneic or xenogeneic with respect to the recipient.

The procedure for ex vivo expansion of hematopoietic stem and progenitor cells is described in U.S. Pat. No. 5,199,942, incorporated herein by reference, can be applied to the cells of the present invention. Other suitable methods are known in the art, therefore the present invention is not limited to any particular method of ex vivo expansion of the cells. Briefly, ex vivo culture and expansion of T cells comprises: (1) collecting CD34+ hematopoietic stem and progenitor cells from a mammal from peripheral blood harvest or bone marrow explants; and (2) expanding such cells ex vivo. In addition to the cellular growth factors described in U.S. Pat. No. 5,199,942, other factors such as flt3-L, IL-1, IL-3 and c-kit ligand, can be used for culturing and expansion of the cells. In addition to using a cell-based vaccine in terms of ex vivo immunization, the present invention also provides compositions and methods for in vivo immunization to elicit an immune response directed against an antigen in a patient.

Generally, the cells activated and expanded as described herein may be utilized in the treatment and prevention of diseases that arise in individuals who are immunocompromised, such as individuals having cancer.

The CAR-modified immune cells (e.g., CAR T cells) of the present invention may be administered either alone, or as a composition (e.g., a pharmaceutical composition) in combination with diluents and/or with other components such as IL-2 or other cytokines or cell populations. Briefly, pharmaceutical compositions of the present invention may comprise a target cell population as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants {e.g., aluminum hydroxide); and preservatives.

Compositions of the present invention are preferably formulated for intravenous administration.

Pharmaceutical compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials.

When “therapeutic amount” is indicated, the precise amount of the compositions of the present invention to be administered can be determined by a physician with consideration of individual differences in age, weight, tumor size, extent of infection or metastasis, and condition of the patient (subject). It can generally be stated that a pharmaceutical composition comprising the CAR-modified immune cells (e.g., CAR T cells) described herein may be administered at a dosage of 10⁴ to 10⁹ cells/kg body weight, preferably 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

In certain embodiments, it may be desired to administer activated immune (e.g., T cells) to a subject and then subsequently redraw blood (or have an apheresis performed), activate T cells therefrom according to the present invention, and reinfuse the patient with these activated and expanded T cells. This process can be carried out multiple times every few weeks. In certain embodiments, T cells can be activated from blood draws of from 10 cc to 400 cc. In certain embodiments, T cells are activated from blood draws of 20 cc, 30 cc, 40 cc, 50 cc, 60 cc, 70 cc, 80 cc, 90 cc, or 100 cc. Not to be bound by theory, using this multiple blood draw/multiple reinfusion protocol may serve to select out certain populations of T cells.

The administration of the subject compositions may be carried out in any convenient manner, including by aerosol inhalation, injection, ingestion, transfusion, implantation or transplantation. The compositions described herein may be administered to a patient subcutaneously, intradermally, intratumorally, intranodally, intramedullary, intramuscularly, by intravenous (i.v.) injection, or intraperitoneally. In some embodiments, the T cell compositions of the present invention are administered to a patient by intradermal or subcutaneous injection. In another embodiment, the T cell compositions of the present invention are preferably administered by i.v. injection. The compositions of T cells may be injected directly into a tumor, lymph node, or site of infection.

In certain embodiments of the present invention, cells activated and expanded using the methods described herein, or other methods known in the art where T cells are expanded to therapeutic levels, are administered to a patient in conjunction with (e.g., before, simultaneously or following) any number of relevant treatment modalities, including but not limited to treatment with agents such as antiviral therapy, cidofovir and interleukin-2, Cytarabine (also known as ARA-C) or natalizumab treatment for MS patients or efalizumab treatment for psoriasis patients or other treatments for PML patients. In further embodiments, the T cells of the invention may be used in combination with chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludaribine, cyclosporin, FK506, rapamycin, mycophenolic acid, steroids, FR901228, cytokines, and irradiation. These drugs inhibit either the calcium dependent phosphatase calcineurin (cyclosporine and FK506) or inhibit the p70S6 kinase that is important for growth factor induced signaling (rapamycin). In a further embodiment, the cell compositions of the present invention are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In another embodiment, the cell compositions of the present invention are administered following B-cell ablative therapy such as agents that react with CD20, e.g., Rituxan. For example, in some embodiments, subjects may undergo standard treatment with high dose chemotherapy followed by peripheral blood stem cell transplantation. In certain embodiments, following the transplant, subjects receive an infusion of the expanded immune cells of the present invention. In an additional embodiment, expanded cells are administered before or following surgery.

In certain embodiments of the present invention, cells activated and expanded using the methods described herein, or other methods known in the art where T cells are expanded to therapeutic levels, or any other compositions described herein, are administered to a patient in conjunction with (e.g., before, simultaneously or following) any number of relevant treatment modalities, including checkpoint inhibitors, such as PD-L1 inhibitors or PD1 inhibitors. In some embodiments, the PD-L1 inhibitors or PD1 inhibitors are PD-L1-specific antibodies or PD1-specific antibodies. Exemplary checkpoint inhibitors include, e.g., pembrolizumab (Merck), ipilimumab (Bristol-Myers Squibb), nivolumab (Bristol-Myers Squibb), MPDL3280A (Roche), MEDI4736 (AstraZeneca), MEDI0680 (AstraZeneca), BMS-936559/MDX-1105 (Bristol-Myers Squibb) and MSB0010718C (Merck). Other PD-L1 and PD1 inhibitors are known in the art (see, e.g., Dolan et al. PD-1 pathway inhibitors: changing the landscape of cancer immunotherapy. Cancer Control. 2014 July; 21(3):231-7). In some embodiments, compositions described herein are administered in conjunction with (e.g., before, simultaneously or following) chemotherapy and/or radiotherapy.

The dosage of the above treatments to be administered to a patient will vary with the precise nature of the condition being treated and the recipient of the treatment. The scaling of dosages for human administration can be performed according to art-accepted practices. The dose for CAMPATH, for example, will generally be in the range 1 to about 100 mg for an adult patient, usually administered daily for a period between 1 and 30 days. The preferred daily dose is 1 to 10 mg per day although in some instances larger doses of up to 40 mg per day may be used (described in U.S. Pat. No. 6,120,766). Strategies for CAR T cell dosing and scheduling have been discussed (Ertl et al, 2011, Cancer Res, 71:3175-81; Junghans, 2010, Journal of Translational Medicine, 8:55).

### EMBODIMENTS

Some embodiments of the invention are as follows:
1. A cell therapy product comprising: a plurality of T cells, wherein at least 80 percent of the T cells are CD8+ cells, wherein at least some of the CD8+ cells express a chimeric antigen receptor protein, wherein the protein comprises an antigen recognition moiety and a T cell activation moiety, and wherein the antigen recognition moiety binds to a B cell malignancy-associated antigen.
2. The product of Embodiment 1, wherein the T cells are essentially free of CD4+ cells.
3. The product of Embodiment 1, wherein at least 80 percent of the CD8+ cells express the chimeric antigen receptor protein.
4. The product of Embodiment 3, wherein at least 90 percent of the CD8+ cells express the chimeric antigen receptor protein.
5. The product of Embodiment 4, wherein at least 85 percent of T cells are CD8+ cells.
6. The product of Embodiment 5, wherein at least 90 percent of T cells are CD8+ cells.
7. The product of Embodiment 6, wherein at least 93 percent of T cells are CD8+ cells.
8. The product of Embodiment 7, wherein at least 95 percent of T cells are CD8+ cells.
9. The product of Embodiment 8, wherein at least 97 percent of T cells are CD8+ cells.
10. The product of Embodiment 9, wherein at least 98 percent of T cells are CD8+ cells.
11. The product of Embodiment 10, wherein at least 99 percent of T cells are CD8+ cells.
12. The product of Embodiment 11, wherein at least 99.5 percent of T cells are CD8+ cells.
13. The product of Embodiment 12, wherein at least 99.9 percent of T cells are CD8+ cells.
14. The product of any one of Embodiments 1-13, wherein at least some of the CD8+ cells comprise mRNA that encodes the chimeric antigen receptor.
15. The product of any one of Embodiments 1-13, wherein the product is a final product suitable for administration to a human.
16. The product of Embodiment 14, wherein the T cell activation moiety comprises a T cell signaling domain selected from the group consisting of: a human CD8-alpha protein, a human CD28 protein, a human CD3-zeta protein, a human FcRγ protein, a CD27 protein, an OX40 protein, a human 4-1BB protein, and modified version any of the foregoing.
17. The product of Embodiment 15, wherein the T cell activation moiety comprises a T cell signaling domain selected from the group consisting of: a human CD8-alpha protein, a human CD28 protein, a human CD3-zeta protein, a human FcRγ protein, a CD27 protein, an OX40 protein, a human 4-1BB protein, and modified version any of the foregoing.
18. The product of Embodiment 14, wherein the B cell malignancy-associated antigen is BCMA.
19. The product of Embodiment 15, wherein the B cell malignancy-associated antigen is BCMA.
20. The product of Embodiment 18, wherein the antigen recognition moiety comprises a variable region of a monoclonal antibody.
21. The product of Embodiment 18, wherein the antigen recognition moiety comprises the (i) heavy chain complementarity determining region (CDR)1, (ii) heavy chain CDR2, (iii) heavy chain CDR3, (iv) light chain CDR1, (v) light chain CDR2, and (vi) light chain CDR3 of one amino acid sequence selected from the group consisting of SEQ ID NO: 4, SEQ ID NO: 5, SEQ ID NO: 6, SEQ ID NO: 8, SEQ ID NO: 9, SEQ ID NO: 10, SEQ ID NO: 11, and SEQ ID NO: 12.
22. The product of Embodiment 18, wherein the antigen recognition moiety comprises the (i) heavy chain variable region and (ii) light chain variable region of one amino acid sequence selected from the group consisting of SEQ ID NO: 4, SEQ ID NO: 5, SEQ ID NO: 6, SEQ ID NO: 8, SEQ ID NO: 9, SEQ ID NO: 10, SEQ ID NO: 11, and SEQ ID NO: 12.
23. The product of Embodiment 19, wherein the antigen recognition moiety comprises the (i) heavy chain complementarity determining region (CDR)1, (ii) heavy chain CDR2, (iii) heavy chain CDR3, (iv) light chain CDR1, (v) light chain CDR2, and (vi) light chain CDR3 of one amino acid sequence selected from the group consisting of SEQ ID NO: 4, SEQ ID NO: 5, SEQ ID NO: 6, SEQ ID NO: 8, SEQ ID NO: 9, SEQ ID NO: 10, SEQ ID NO: 11, and SEQ ID NO: 12.
24. The product of Embodiment 19, wherein the antigen recognition moiety comprises the (i) heavy chain variable region and (ii) light chain variable region of one amino acid sequence selected from the group consisting of SEQ ID NO: 4, SEQ ID NO: 5, SEQ ID NO: 6, SEQ ID NO: 8, SEQ ID NO: 9, SEQ ID NO: 10, SEQ ID NO: 11, and SEQ ID NO: 12.
25. The product of any one of Embodiments 1-13, wherein the B cell malignancy-associated antigen is BCMA.
26. The product of Embodiment 25, wherein the antigen recognition moiety comprises a variable region of a monoclonal antibody.
27. The product of Embodiment 26, wherein the antigen recognition moiety comprises the (i) heavy chain complementarity determining region (CDR)1, (ii) heavy chain CDR2, (iii) heavy chain CDR3, (iv) light chain CDR1, (v) light chain CDR2, and (vi) light chain CDR3 of one amino acid sequence selected from the group consisting of SEQ ID NO: 4, SEQ ID NO: 5, SEQ ID NO: 6, SEQ ID NO: 8, SEQ ID NO: 9, SEQ ID NO: 10, SEQ ID NO: 11, and SEQ ID NO: 12.
28. The product of Embodiment 27, wherein the antigen recognition moiety comprises the (i) heavy chain variable region and (ii) light chain variable region of one amino acid sequence selected from the group consisting of SEQ ID NO: 4, SEQ ID NO: 5, SEQ ID NO: 6, SEQ ID NO: 8, SEQ ID NO: 9, SEQ ID NO: 10, SEQ ID NO: 11, and SEQ ID NO: 12.
29. The product of Embodiment 25, wherein the T cell activation moiety comprises a T cell signaling domain selected from the group consisting of: a human CD8-alpha protein, a human CD28 protein, a human CD3-zeta protein, a human FcRγ protein, a CD27 protein, an OX40 protein, a human 4-1BB protein, and modified version any of the foregoing.
30. The product of Embodiment 25, wherein the T cell activation moiety comprises a T cell signaling domain selected from the group consisting of: a human CD8-alpha protein, a human CD28 protein, a human CD3-zeta protein, a human FcRγ protein, a CD27 protein, an OX40 protein, a human 4-1BB protein, and modified version any of the foregoing.
31. A method for producing a cell therapy product, the method comprising: purifying CD8+

T cells; and transfecting the CD8+ T cells with a synthetic nucleic acid construct encoding a chimeric antigen receptor protein, wherein the protein comprises an antigen recognition moiety and a T cell activation moiety, and wherein the antigen recognition moiety binds to a B cell malignancy-associated antigen.
32. The method of Embodiment 31, wherein the product is essentially free of CD4+ cells.
33. The method of Embodiment 31, wherein at least 80 percent of the CD8+ cells express the chimeric antigen receptor protein.
34. The method of Embodiment 33, wherein at least 90 percent of the CD8+ cells express the chimeric antigen receptor protein.
35. The method of Embodiment 31, wherein at least 80 percent of T cells are CD8+ cells.
36. The method of Embodiment 35, wherein at least 90 percent of T cells are CD8+ cells.
37. The method of Embodiment 36, wherein at least 93 percent of T cells are CD8+ cells.
38. The method of Embodiment 37, wherein at least 95 percent of T cells are CD8+ cells.
39. The method of Embodiment 38, wherein at least 97 percent of T cells are CD8+ cells.
40. The method of Embodiment 39, wherein at least 98 percent of T cells are CD8+ cells.
41. The method of Embodiment 40, wherein at least 99 percent of T cells are CD8+ cells.
42. The method of Embodiment 41, wherein at least 99.5 percent of T cells are CD8+ cells.
43. The method of Embodiment 42, wherein at least 99.9 percent of T cells are CD8+ cells.
44. The method of any one of Embodiments 31-43, wherein the nucleic acid construct comprises mRNA.
45. The method of any one of Embodiments 31-43, wherein the product is a final product suitable for human use.
46. The method of Embodiment 44, wherein the T cell activation moiety comprises a T cell signaling domain selected from the group consisting of: a human CD8-alpha protein, a human CD28 protein, a human CD3-zeta protein, a human FcRγ protein, a CD27 protein, an OX40 protein, a human 4-1BB protein, and modified version any of the foregoing.
47. The method of Embodiment 45, wherein the T cell activation moiety comprises a T cell signaling domain selected from the group consisting of: a human CD8-alpha protein, a human CD28 protein, a human CD3-zeta protein, a human FcRγ protein, a CD27 protein, an OX40 protein, a human 4-1BB protein, and modified version any of the foregoing.
48. The method of Embodiment 44, wherein the B cell malignancy-associated antigen is BCMA.
49. The method of Embodiment 45, wherein the B cell malignancy-associated antigen is BCMA.
50. The method of Embodiment 48, wherein the antigen recognition moiety comprises a variable region of a monoclonal antibody.
51. The method of Embodiment 48, wherein the antigen recognition moiety comprises the (i) heavy chain complementarity determining region (CDR)1, (ii) heavy chain CDR2, (iii) heavy chain CDR3, (iv) light chain CDR1, (v) light chain CDR2, and (vi) light chain CDR3 of one amino acid sequence selected from the group consisting of SEQ ID NO: 4, SEQ ID NO: 5, SEQ ID NO: 6, SEQ ID NO: 8, SEQ ID NO: 9, SEQ ID NO: 10, SEQ ID NO: 11, and SEQ ID NO: 12.
52. The method of Embodiment 48, wherein the antigen recognition moiety comprises the (i) heavy chain variable region and (ii) light chain variable region of one amino acid sequence selected from the group consisting of SEQ ID NO: 4, SEQ ID NO: 5, SEQ ID NO: 6, SEQ ID NO: 8, SEQ ID NO: 9, SEQ ID NO: 10, SEQ ID NO: 11, and SEQ ID NO: 12.
53. The method of Embodiment 49, wherein the antigen recognition moiety comprises the (i) heavy chain complementarity determining region (CDR)1, (ii) heavy chain CDR2, (iii) heavy chain CDR3, (iv) light chain CDR1, (v) light chain CDR2, and (vi) light chain CDR3 of one amino acid sequence selected from the group consisting of SEQ ID NO: 4, SEQ ID NO: 5, SEQ ID NO: 6, SEQ ID NO: 8, SEQ ID NO: 9, SEQ ID NO: 10, SEQ ID NO: 11, and SEQ ID NO: 12.
54. The method of Embodiment 49, wherein the antigen recognition moiety comprises the (i) heavy chain variable region and (ii) light chain variable region of one amino acid sequence selected from the group consisting of SEQ ID NO: 4, SEQ ID NO: 5, SEQ ID NO: 6, SEQ ID NO: 8, SEQ ID NO: 9, SEQ ID NO: 10, SEQ ID NO: 11, and SEQ ID NO: 12.
55. The method of any one of Embodiments 31-43, wherein the B cell malignancy-associated antigen is BCMA.
56. The method of Embodiment 55, wherein the antigen recognition moiety comprises a variable region of a monoclonal antibody.
57. The method of Embodiment 56, wherein the antigen recognition moiety comprises the (i) heavy chain complementarity determining region (CDR)1, (ii) heavy chain CDR2, (iii) heavy chain CDR3, (iv) light chain CDR1, (v) light chain CDR2, and (vi) light chain CDR3 of one amino acid sequence selected from the group consisting of SEQ ID NO: 4, SEQ ID NO: 5, SEQ ID NO: 6, SEQ ID NO: 8, SEQ ID NO: 9, SEQ ID NO: 10, SEQ ID NO: 11, and SEQ ID NO: 12.
58. The method of Embodiment 57, wherein the antigen recognition moiety comprises the (i) heavy chain variable region and (ii) light chain variable region of one amino acid sequence selected from the group consisting of SEQ ID NO: 4, SEQ ID NO: 5, SEQ ID NO: 6, SEQ ID NO: 8, SEQ ID NO: 9, SEQ ID NO: 10, SEQ ID NO: 11, and SEQ ID NO: 12.
59. The method of Embodiment 55, wherein the T cell activation moiety comprises a T cell signaling domain selected from the group consisting of: a human CD8-alpha protein, a human CD28 protein, a human CD3-zeta protein, a human FcRγ protein, a CD27 protein, an OX40 protein, a human 4-1BB protein, and modified version any of the foregoing.
60. The method of Embodiment 55, wherein the T cell activation moiety comprises a T cell signaling domain selected from the group consisting of: a human CD8-alpha protein, a human CD28 protein, a human CD3-zeta protein, a human FcRγ protein, a CD27 protein, an OX40 protein, a human 4-1BB protein, and modified version any of the foregoing.
61. The method of any one of Embodiments 31-43, wherein the purifying comprises negative selection.
62. The method of any one of Embodiments 31-43, wherein the purifying comprises positive selection.
63. The method of any one of Embodiments 31-43, wherein the transfecting comprises electroporation.
64. A method for producing a cell therapy product, the method comprising: transfecting T cells with a synthetic nucleic acid construct encoding a chimeric antigen receptor protein, wherein the chimeric antigen receptor protein comprises an antigen recognition moiety and a T cell activation moiety, and wherein the antigen recognition moiety binds to a B cell malignancy-associated antigen; and purifying CD8+ cells from the transfected T cells.
65. The method of Embodiment 64, wherein the final product is essentially free of CD4+ cells.
66. The method of Embodiment 64, wherein at least 80 percent of the CD8+ cells express the chimeric antigen receptor protein.
67. The method of Embodiment 66, wherein at least 90 percent of the CD8+ cells express the chimeric antigen receptor protein.
68. The product of Embodiment 64, wherein at least 80 percent of T cells in the final product are CD8+ cells. 69. The method of Embodiment 68, wherein at least 90 percent of T cells in the final product are CD8+ cells.
70. The method of Embodiment 69, wherein at least 93 percent of T cells in the final product are CD8+ cells.
71. The method of Embodiment 70, wherein at least 95 percent of T cells in the final product are CD8+ cells.
72. The method of Embodiment 71, wherein at least 97 percent of T cells in the final product are CD8+ cells.
73. The method of Embodiment 72, wherein at least 98 percent of T cells in the final product are CD8+ cells.
74. The method of Embodiment 73, wherein at least 99 percent of T cells in the final product are CD8+ cells.
75. The method of Embodiment 74, wherein at least 99.5 percent of T cells in the final product are CD8+ cells.
76. The method of Embodiment 75, wherein at least 99.9 percent of T cells in the final product are CD8+ cells.
77. The method of any one of Embodiments 64-76, wherein the nucleic acid construct comprises mRNA.
78. The method of any one of Embodiments 64-76, wherein the product is a final product suitable for human use.
79. The method of Embodiment 77, wherein the T cell activation moiety comprises a T cell signaling domain selected from the group consisting of: a human CD8-alpha protein, a human CD28 protein, a human CD3-zeta protein, a human FcRγ protein, a CD27 protein, an OX40 protein, a human 4-1BB protein, and modified version any of the foregoing.
80. The method of Embodiment 78, wherein the T cell activation moiety comprises a T cell signaling domain selected from the group consisting of: a human CD8-alpha protein, a human CD28 protein, a human CD3-zeta protein, a human FcRγ protein, a CD27 protein, an OX40 protein, a human 4-1BB protein, and modified version any of the foregoing.
81. The method of Embodiment 77, wherein the B cell malignancy-associated antigen is BCMA.
82. The method of Embodiment 78, wherein the B cell malignancy-associated antigen is BCMA.
83. The method of Embodiment 81, wherein the antigen recognition moiety comprises a variable region of a monoclonal antibody.
84. The method of Embodiment 81, wherein the antigen recognition moiety comprises the (i) heavy chain complementarity determining region (CDR)1, (ii) heavy chain CDR2, (iii) heavy chain CDR3, (iv) light chain CDR1, (v) light chain CDR2, and (vi) light chain CDR3 of one amino acid sequence selected from the group consisting of SEQ ID NO: 4, SEQ ID NO: 5, SEQ ID NO: 6, SEQ ID NO: 8, SEQ ID NO: 9, SEQ ID NO: 10, SEQ ID NO: 11, and SEQ ID NO: 12.
85. The method of Embodiment 81, wherein the antigen recognition moiety comprises the (i) heavy chain variable region and (ii) light chain variable region of one amino acid sequence selected from the group consisting of SEQ ID NO: 4, SEQ ID NO: 5, SEQ ID NO: 6, SEQ ID NO: 8, SEQ ID NO: 9, SEQ ID NO: 10, SEQ ID NO: 11, and SEQ ID NO: 12.
86. The method of Embodiment 82, wherein the antigen recognition moiety comprises the (i) heavy chain complementarity determining region (CDR)1, (ii) heavy chain CDR2, (iii) heavy chain CDR3, (iv) light chain CDR1, (v) light chain CDR2, and (vi) light chain CDR3 of one amino acid sequence selected from the group consisting of SEQ ID NO: 4, SEQ ID NO: 5, SEQ ID NO: 6, SEQ ID NO: 8, SEQ ID NO: 9, SEQ ID NO: 10, SEQ ID NO: 11, and SEQ ID NO: 12.
87. The method of Embodiment 82, wherein the antigen recognition moiety comprises the (i) heavy chain variable region and (ii) light chain variable region of one amino acid sequence selected from the group consisting of SEQ ID NO: 4, SEQ ID NO: 5, SEQ ID NO: 6, SEQ ID NO: 8, SEQ ID NO: 9, SEQ ID NO: 10, SEQ ID NO: 11, and SEQ ID NO: 12.
88. The method of any one of Embodiments 64-76, wherein the B cell malignancy-associated antigen is BCMA.
89. The method of Embodiment 88, wherein the antigen recognition moiety comprises a variable region of a monoclonal antibody.
90. The method of Embodiment 89, wherein the antigen recognition moiety comprises the (i) heavy chain complementarity determining region (CDR)1, (ii) heavy chain CDR2, (iii) heavy chain CDR3, (iv) light chain CDR1, (v) light chain CDR2, and (vi) light chain CDR3 of one amino acid sequence selected from the group consisting of SEQ ID NO: 4, SEQ ID NO: 5, SEQ ID NO: 6, SEQ ID NO: 8, SEQ ID NO: 9, SEQ ID NO: 10, SEQ ID NO: 11, and SEQ ID NO: 12.
91. The method of Embodiment 90, wherein the antigen recognition moiety comprises the (i) heavy chain variable region and (ii) light chain variable region of one amino acid sequence selected from the group consisting of SEQ ID NO: 4, SEQ ID NO: 5, SEQ ID NO: 6, SEQ ID NO: 8, SEQ ID NO: 9, SEQ ID NO: 10, SEQ ID NO: 11, and SEQ ID NO: 12.
92. The method of Embodiment 88, wherein the T cell activation moiety comprises a T cell signaling domain selected from the group consisting of: a human CD8-alpha protein, a human CD28 protein, a human CD3-zeta protein, a human FcRγ protein, a CD27 protein, an OX40 protein, a human 4-1BB protein, and modified version any of the foregoing. 93. The method of Embodiment 88, wherein the T cell activation moiety comprises a T cell signaling domain selected from the group consisting of: a human CD8-alpha protein, a human CD28 protein, a human CD3-zeta protein, a human FcRγ protein, a CD27 protein, an OX40 protein, a human 4-1BB protein, and modified version any of the foregoing.
94. The method of any one of Embodiments 64-76, wherein the purifying comprises negative selection.
95. The method of any one of Embodiments 64-76, wherein the purifying comprises positive selection.
96. The method of any one of Embodiments 64-76, wherein the transfecting comprises electroporation.
97. A method for treating a B cell malignancy in an individual, the method comprising administering to the individual a product of any one of Embodiments 1-13.
98. A method for treating a B cell malignancy in an individual, the method comprising administering to the individual a product of Embodiment 14.
99. A method for treating a B cell malignancy in an individual, the method comprising administering to the individual a product of Embodiment 15.
100. A method for treating a B cell malignancy in an individual, the method comprising administering to the individual a product of Embodiment 18.
101. A method for treating a B cell malignancy in an individual, the method comprising administering to the individual a product of Embodiment 19.
102. A method for treating a B cell malignancy in an individual, the method comprising administering to the individual a product of Embodiment 25. 103. The method of Embodiment 97, wherein the individual suffers from multiple myeloma.
104. The method of Embodiment 98, wherein the individual suffers from multiple myeloma.
105. The method of Embodiment 99, wherein the individual suffers from multiple myeloma.
106. The method of Embodiment 100, wherein the individual suffers from multiple myeloma.
107. The method of Embodiment 101, wherein the individual suffers from multiple myeloma.
108. The method of Embodiment 102, wherein the individual suffers from multiple myeloma.
109. A method for treating a B cell malignancy in an individual, the method comprising administering to the individual a product prepared according to the method of any one of Embodiments 31-43.
110. A method for treating a B cell malignancy in an individual, the method comprising administering to the individual a product prepared according to the method of Embodiment 44.
111. A method for treating a B cell malignancy in an individual, the method comprising administering to the individual a product prepared according to the method of Embodiment 45.
112. A method for treating a B cell malignancy in an individual, the method comprising administering to the individual a product prepared according to the method of Embodiment 48.
113. A method for treating a B cell malignancy in an individual, the method comprising administering to the individual a product prepared according to the method of Embodiment 49.
114. A method for treating a B cell malignancy in an individual, the method comprising administering to the individual a product prepared according to the method of Embodiment 55.
115. The method of Embodiment 109, wherein the individual suffers from multiple myeloma.
116. The method of Embodiment 110, wherein the individual suffers from multiple myeloma.
117. The method of Embodiment 111, wherein the individual suffers from multiple myeloma.
118. The method of Embodiment 112, wherein the individual suffers from multiple myeloma.
119. The method of Embodiment 113, wherein the individual suffers from multiple myeloma.
120. The method of Embodiment 114, wherein the individual suffers from multiple myeloma.
121. A method for treating a B cell malignancy in an individual, the method comprising administering to the individual a product prepared according to the method of any one of Embodiments 64-76.
122. A method for treating a B cell malignancy in an individual, the method comprising administering to the individual a product prepared according to the method of Embodiment 77.
123. A method for treating a B cell malignancy in an individual, the method comprising administering to the individual a product prepared according to the method of Embodiment 78.
124. A method for treating a B cell malignancy in an individual, the method comprising administering to the individual a product prepared according to the method of Embodiment 81.
125. A method for treating a B cell malignancy in an individual, the method comprising administering to the individual a product prepared according to the method of Embodiment 82.
126. A method for treating a B cell malignancy in an individual, the method comprising administering to the individual a product prepared according to the method of Embodiment 88.
127. The method of Embodiment 121, wherein the individual suffers from multiple myeloma.
128. The method of Embodiment 122, wherein the individual suffers from multiple myeloma.
129. The method of Embodiment 123, wherein the individual suffers from multiple myeloma.
130. The method of Embodiment 124, wherein the individual suffers from multiple myeloma.
131. The method of Embodiment 125, wherein the individual suffers from multiple myeloma.
132. The method of Embodiment 126, wherein the individual suffers from multiple myeloma.
133. A method for treating a B cell malignancy in an individual, the method comprising: collecting blood cells from the individual; purifying CD8+ T cells from the blood cells; transfecting the CD8+ T cells with a synthetic nucleic construct encoding a chimeric antigen receptor protein, wherein the protein comprises an antigen recognition moiety and a T cell activation moiety, and wherein the antigen recognition moiety binds to a B cell malignancy-associated antigen; and administering the transfected CD8+ cells to the individual.
134. The method of Embodiment 133, wherein the final product is essentially free of CD4+ cells.
135. The method of Embodiment 133, wherein at least 80 percent of the CD8+ cells express the chimeric antigen receptor protein.
136. The method of Embodiment 135, wherein at least 90 percent of the CD8+ cells express the chimeric antigen receptor protein.
137. The method of Embodiment 133, wherein at least 80 percent of T cells in the final product are CD8+ cells.
138. The method of Embodiment 137, wherein at least 90 percent of T cells in the final product are CD8+ cells.
139. The method of Embodiment 138, wherein at least 93 percent of T cells in the final product are CD8+ cells.
140. The method of Embodiment 139, wherein at least 95 percent of T cells in the final product are CD8+ cells.
141. The method of Embodiment 140, wherein at least 97 percent of T cells in the final product are CD8+ cells.
142. The method of Embodiment 141, wherein at least 98 percent of T cells in the final product are CD8+ cells.
143. The method of Embodiment 142, wherein at least 99 percent of T cells in the final product are CD8+ cells.
144. The method of Embodiment 143, wherein at least 99.5 percent of T cells in the final product are CD8+ cells.
145. The method of Embodiment 144, wherein at least 99.9 percent of T cells in the final product are CD8+ cells.
146. The method of any one of Embodiments 133-145, wherein the nucleic acid construct comprises mRNA.
147. The method of any one of Embodiments 133-145, wherein the product is a final product suitable for human use.
148. The method of Embodiment 146, wherein the T cell activation moiety comprises a T cell signaling domain selected from the group consisting of: a human CD8-alpha protein, a human CD28 protein, a human CD3-zeta protein, a human FcRγ protein, a CD27 protein, an OX40 protein, a human 4-1BB protein, and modified version any of the foregoing.
149. The method of Embodiment 147, wherein the T cell activation moiety comprises a T cell signaling domain selected from the group consisting of: a human CD8-alpha protein, a human CD28 protein, a human CD3-zeta protein, a human FcRγ protein, a CD27 protein, an OX40 protein, a human 4-1BB protein, and modified version any of the foregoing.
150. The method of Embodiment 146, wherein the B cell malignancy-associated antigen is BCMA.
151. The method of Embodiment 147, wherein the B cell malignancy-associated antigen is BCMA.
152. The method of Embodiment 150, wherein the antigen recognition moiety comprises a variable region of a monoclonal antibody.
153. The method of Embodiment 150, wherein the antigen recognition moiety comprises the (i) heavy chain complementarity determining region (CDR)1, (ii) heavy chain CDR2, (iii) heavy chain CDR3, (iv) light chain CDR1, (v) light chain CDR2, and (vi) light chain CDR3 of one amino acid sequence selected from the group consisting of SEQ ID NO: 4, SEQ ID NO: 5, SEQ ID NO: 6, SEQ ID NO: 8, SEQ ID NO: 9, SEQ ID NO: 10, SEQ ID NO: 11, and SEQ ID NO: 12.
154. The method of Embodiment 150, wherein the antigen recognition moiety comprises the (i) heavy chain variable region and (ii) light chain variable region of one amino acid sequence selected from the group consisting of SEQ ID NO: 4, SEQ ID NO: 5, SEQ ID NO: 6, SEQ ID NO: 8, SEQ ID NO: 9, SEQ ID NO: 10, SEQ ID NO: 11, and SEQ ID NO: 12.
155. The method of Embodiment 151, wherein the antigen recognition moiety comprises the (i) heavy chain complementarity determining region (CDR)1, (ii) heavy chain CDR2, (iii) heavy chain CDR3, (iv) light chain CDR1, (v) light chain CDR2, and (vi) light chain CDR3 of one amino acid sequence selected from the group consisting of SEQ ID NO: 4, SEQ ID NO: 5, SEQ ID NO: 6, SEQ ID NO: 8, SEQ ID NO: 9, SEQ ID NO: 10, SEQ ID NO: 11, and SEQ ID NO: 12.
156. The method of Embodiment 151, wherein the antigen recognition moiety comprises the (i) heavy chain variable region and (ii) light chain variable region of one amino acid sequence selected from the group consisting of SEQ ID NO: 4, SEQ ID NO: 5, SEQ ID NO: 6, SEQ ID NO: 8, SEQ ID NO: 9, SEQ ID NO: 10, SEQ ID NO: 11, and SEQ ID NO: 12.
157. The method of any one of Embodiments 133-145, wherein the individual suffers from multiple myeloma.
158. The method of any one of Embodiments 133-145, wherein the B cell malignancy-associated antigen is BCMA.
159. The method of Embodiment 157, wherein the antigen recognition moiety comprises a variable region of a monoclonal antibody.
160. The method of Embodiment 158, wherein the antigen recognition moiety comprises the (i) heavy chain complementarity determining region (CDR)1, (ii) heavy chain CDR2, (iii) heavy chain CDR3, (iv) light chain CDR1, (v) light chain CDR2, and (vi) light chain CDR3 of one amino acid sequence selected from the group consisting of SEQ ID NO: 4, SEQ ID NO: 5, SEQ ID NO: 6, SEQ ID NO: 8, SEQ ID NO: 9, SEQ ID NO: 10, SEQ ID NO: 11, and SEQ ID NO: 12.
161. The method of Embodiment 159, wherein the antigen recognition moiety comprises the (i) heavy chain variable region and (ii) light chain variable region of one amino acid sequence selected from the group consisting of SEQ ID NO: 4, SEQ ID NO: 5, SEQ ID NO: 6, SEQ ID NO: 8, SEQ ID NO: 9, SEQ ID NO: 10, SEQ ID NO: 11, and SEQ ID NO: 12.
162. The method of Embodiment 151, wherein the T cell activation moiety comprises a T cell signaling domain selected from the group consisting of: a human CD8-alpha protein, a human CD28 protein, a human CD3-zeta protein, a human FcRγ protein, a CD27 protein, an OX40 protein, a human 4-1BB protein, and modified version any of the foregoing.
163. The method of Embodiment 157, wherein the T cell activation moiety comprises a T cell signaling domain selected from the group consisting of: a human CD8-alpha protein, a human CD28 protein, a human CD3-zeta protein, a human FcRγ protein, a CD27 protein, an OX40 protein, a human 4-1BB protein, and modified version any of the foregoing.
164. The method of any one of Embodiments 133-145, wherein the purifying comprises negative selection.
165. The method of any one of Embodiments 133-145, wherein the purifying comprises positive selection.
166. The method of any one of Embodiments 133-145, wherein the transfecting comprises electroporation.
167. The method of any one of Embodiments 133-145, wherein the method is a first-line therapy.
168. The method of any one of Embodiments 133-145, wherein the individual has not undergone a bone marrow transplant.
169. A method for treating a B cell malignancy in an individual, the method comprising:

collecting blood cells from the individual; purifying CD8+ T cells from the blood cells;

transfecting the CD8+ T cells with a synthetic nucleic acid construct encoding a chimeric antigen receptor protein, wherein the protein comprises an antigen recognition moiety and a T cell activation moiety, and wherein the antigen recognition moiety binds to a B cell malignancy-associated antigen; and administering the transfected CD8+ cells to the individual.
170. The method of Embodiment 169, wherein the final product is essentially free of CD4+ cells.
171. The method of Embodiment 169, wherein at least 80 percent of the CD8+ cells express the chimeric antigen receptor protein.
172. The method of Embodiment 171, wherein at least 90 percent of the CD8+ cells express the chimeric antigen receptor protein.
173. The method of Embodiment 169, wherein at least 80 percent of T cells in the final product are CD8+ cells.
174. The method of Embodiment 173, wherein at least 90 percent of T cells in the final product are CD8+ cells.
175. The method of Embodiment 174, wherein at least 93 percent of T cells in the final product are CD8+ cells.
176. The method of Embodiment 175, wherein at least 95 percent of T cells in the final product are CD8+ cells.
177. The method of Embodiment 176, wherein at least 97 percent of T cells in the final product are CD8+ cells.
178. The method of Embodiment 177, wherein at least 98 percent of T cells in the final product are CD8+ cells.
179. The method of Embodiment 178, wherein at least 99 percent of T cells in the final product are CD8+ cells.
180. The method of Embodiment 179, wherein at least 99.5 percent of T cells in the final product are CD8+ cells.
181. The method of Embodiment 180, wherein at least 99.9 percent of T cells in the final product are CD8+ cells.
182. The method of any one of Embodiments 169-181, wherein the nucleic acid construct comprises mRNA.
183. The method of any one of Embodiments 169-181, wherein the product is a final product suitable for human use.
184. The method of Embodiment 182, wherein the T cell activation moiety comprises a T cell signaling domain selected from the group consisting of: a human CD8-alpha protein, a human CD28 protein, a human CD3-zeta protein, a human FcRγ protein, a CD27 protein, an OX40 protein, a human 4-1BB protein, and modified version any of the foregoing.
185. The method of Embodiment 183, wherein the T cell activation moiety comprises a T cell signaling domain selected from the group consisting of: a human CD8-alpha protein, a human CD28 protein, a human CD3-zeta protein, a human FcRγ protein, a CD27 protein, an OX40 protein, a human 4-1BB protein, and modified version any of the foregoing.
186. The method of Embodiment 182, wherein the B cell malignancy-associated antigen is BCMA.
187. The method of Embodiment 183, wherein the B cell malignancy-associated antigen is BCMA.
188. The method of Embodiment 186, wherein the antigen recognition moiety comprises a variable region of a monoclonal antibody.
189. The method of Embodiment 186, wherein the antigen recognition moiety comprises the (i) heavy chain complementarity determining region (CDR)1, (ii) heavy chain CDR2, (iii) heavy chain CDR3, (iv) light chain CDR1, (v) light chain CDR2, and (vi) light chain CDR3 of one amino acid sequence selected from the group consisting of SEQ ID NO: 4, SEQ ID NO: 5, SEQ ID NO: 6, SEQ ID NO: 8, SEQ ID NO: 9, SEQ ID NO: 10, SEQ ID NO: 11, and SEQ ID NO: 12.
190. The method of Embodiment 186, wherein the antigen recognition moiety comprises the (i) heavy chain variable region and (ii) light chain variable region of one amino acid sequence selected from the group consisting of SEQ ID NO: 4, SEQ ID NO: 5, SEQ ID NO: 6, SEQ ID NO: 8, SEQ ID NO: 9, SEQ ID NO: 10, SEQ ID NO: 11, and SEQ ID NO: 12.
191. The method of Embodiment 187, wherein the antigen recognition moiety comprises the (i) heavy chain complementarity determining region (CDR)1, (ii) heavy chain CDR2, (iii) heavy chain CDR3, (iv) light chain CDR1, (v) light chain CDR2, and (vi) light chain CDR3 of one amino acid sequence selected from the group consisting of SEQ ID NO: 4, SEQ ID NO: 5, SEQ ID NO: 6, SEQ ID NO: 8, SEQ ID NO: 9, SEQ ID NO: 10, SEQ ID NO: 11, and SEQ ID NO: 12.
192. The method of Embodiment 187, wherein the antigen recognition moiety comprises the (i) heavy chain variable region and (ii) light chain variable region of one amino acid sequence selected from the group consisting of SEQ ID NO: 4, SEQ ID NO: 5, SEQ ID NO: 6, SEQ ID NO: 8, SEQ ID NO: 9, SEQ ID NO: 10, SEQ ID NO: 11, and SEQ ID NO: 12.
193. The method of any one of Embodiments 169-181, wherein the B cell malignancy-associated antigen is BCMA.
194. The method of Embodiment 193, wherein the antigen recognition moiety comprises a variable region of a monoclonal antibody.
195. The method of Embodiment 194, wherein the antigen recognition moiety comprises the (i) heavy chain complementarity determining region (CDR)1, (ii) heavy chain CDR2, (iii) heavy chain CDR3, (iv) light chain CDR1, (v) light chain CDR2, and (vi) light chain CDR3 of one amino acid sequence selected from the group consisting of SEQ ID NO: 4, SEQ ID NO: 5, SEQ ID NO: 6, SEQ ID NO: 8, SEQ ID NO: 9, SEQ ID NO: 10, SEQ ID NO: 11, and SEQ ID NO: 12.
196. The method of Embodiment 195, wherein the antigen recognition moiety comprises the (i) heavy chain variable region and (ii) light chain variable region of one amino acid sequence selected from the group consisting of SEQ ID NO: 4, SEQ ID NO: 5, SEQ ID NO: 6, SEQ ID NO: 8, SEQ ID NO: 9, SEQ ID NO: 10, SEQ ID NO: 11, and SEQ ID NO: 12.
197. The method of Embodiment 187, wherein the T cell activation moiety comprises a T cell signaling domain selected from the group consisting of: a human CD8-alpha protein, a human CD28 protein, a human CD3-zeta protein, a human FcRγ protein, a CD27 protein, an OX40 protein, a human 4-1BB protein, and modified version any of the foregoing.
198. The method of Embodiment 193, wherein the T cell activation moiety comprises a T cell signaling domain selected from the group consisting of: a human CD8-alpha protein, a human CD28 protein, a human CD3-zeta protein, a human FcRγ protein, a CD27 protein, an OX40 protein, a human 4-1BB protein, and modified version any of the foregoing.
199. The method of any one of Embodiments 169-181, wherein the purifying comprises negative selection.
200. The method of any one of Embodiments 169-181, wherein the purifying comprises positive selection.
201. The method of any one of Embodiments 169-181, wherein the transfecting comprises electroporation.
202. The method of any one of Embodiments 169-181, wherein the individual suffers from multiple myeloma.
203. The method of any one of Embodiments 169-181, wherein the method is a first-line therapy.
204. The method of any one of Embodiments 169-181, wherein the individual has not undergone a bone marrow transplant.

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example 1

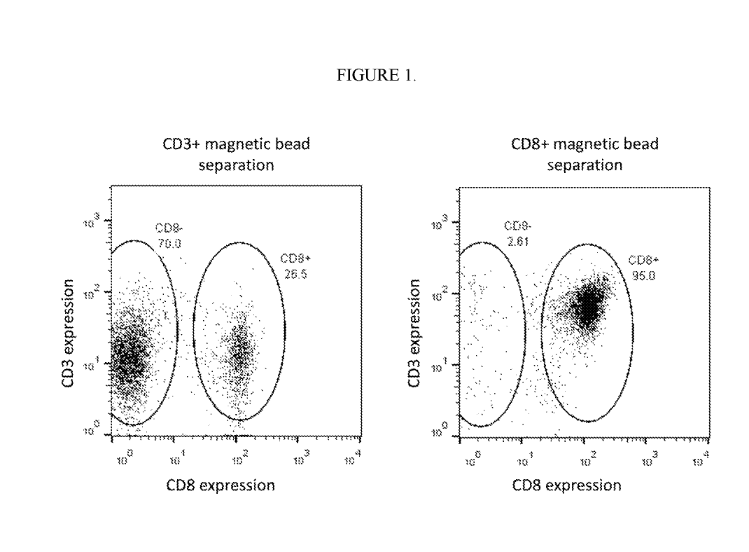

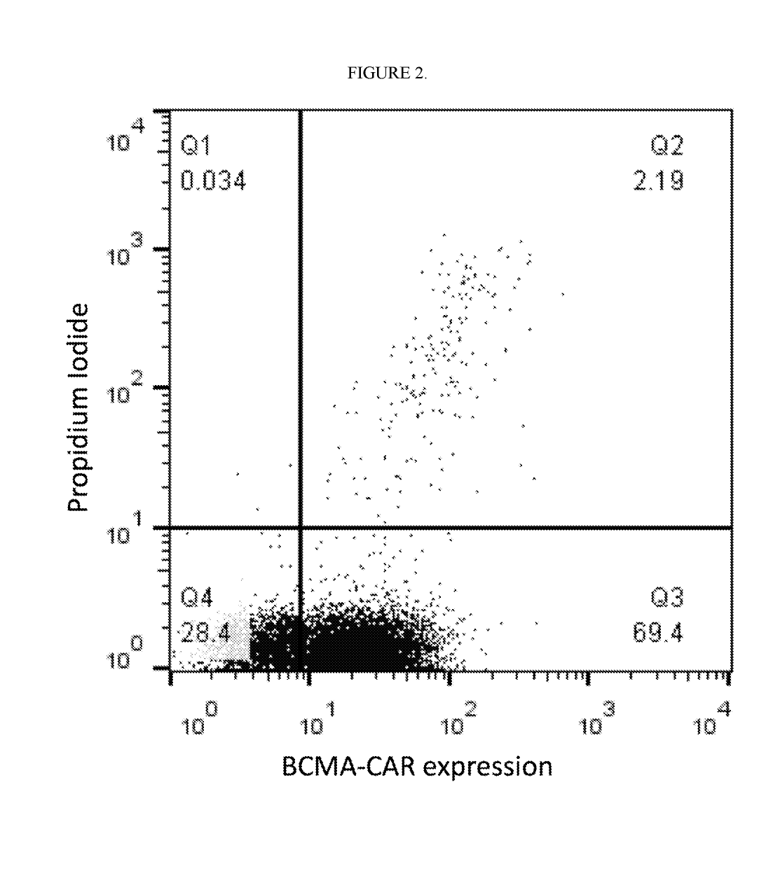

The Following Example Describes Preparation of a Cell Therapy product comprising highly enriched CD8+ CAR T cells that bind BCMA. PBMCs were obtained from donors by phlebotomy followed by FICOLL® density centrifugation. CD8+ T cells were purified by positive selection by incubating cells with paramagnetic CD8 microbeads for 15 min at 4° C., loaded on a MACS® Column, and selected by placing the column in a magnetic field. As an alternative method, CD8+ T cells were purified by negative selection by incubating PBMCs with a paramagnetic bead that bind a heterogeneous group of targets corresponding to non-CD8 T-cells (Stemcell Technologies), column loading, magnetic separation, and elutriation of unbound (CD8+) cells. CD3+ T cells were separated in a similar fashion using CD3 microbeads. Following CD8+ T cell separation, viability of CD8+ T cells was 98%. Over 95% of the total cell population was CD8+ T cells, and over 95% of the CD3+ T cell population was CD8+ T cells (FIG. 1). The purified CD8+ T cells were incubated at 37° C. and then transfected by electroporation (4D Nucleofector, Lonza) with 1.8 μg/cell of mRNA corresponding to SEQ ID: 5, which encodes a CAR that binds BCMA. In some experiments, cells are cultured for at least 1 day prior to transfection in the presence of media supplements (e.g., anti-CD3 antibody, IL-2, and/or IL-15). The cells were incubated overnight at 37° C. with 5% CO₂. Expression of the CAR and binding to BCMA were demonstrated by incubating the cells at a 1:50 dilution with a 200 μg/ml solution of biotinylated BCMA for 30 minutes, washing in a phosphate buffered saline (PBS)-4% bovine serum albumin (BSA) solution, and reincubating with ALEXA FLUOR®-conjugated streptavidin for 15 minutes. Dead cells were stained with propidium iodide. Viability and transfection efficiency were assessed by flow cytometry. Purified, negatively selected CD3+ cells were used as a positive control. Following electroporation, 98% of the CD8+ T cells were viable. About 70% of the purified cell population was CD8+ T cells that expressed the CAR (FIG. 2). Thus, a cell therapy product comprising highly enriched CD8+ CAR T cells was successfully prepared by transfection of an mRNA construct encoding a CAR directed to BCMA.

### Example 2

The following example describes a tumor cytotoxicity assay wherein, in response to a BCMA-expressing tumor, highly enriched CD8+ CAR T cells killed BCMA+ myeloma cells more efficiently than mixed CD8+/CD4+ CAR T cells. Samples of highly enriched CD8+ CAR T cells were prepared according to the methods of Example 1. Mixed CD3+ CAR T cells were prepared by similar transfection techniques on unenriched CD3+ cells. Samples were incubated overnight at 37° C.+5% CO₂ in the presence of a BCMA+ myeloma cell line (MM1.S) that was pre-labeled with a fluorescent viability dye (CFSE) at 37° C.+5% CO₂. Approximately 50,000 labeled tumor cells were incubated with 200,000 CD8+ T cells or CD3+ T cells (i.e., a 4:1 effector:target ratio). Following the incubation, dead cells were stained with propidium iodide. Flow cytometric analysis was used to distinguish tumor cells from unlabeled T cells both by size and fluorescence staining. Observed rates of cell death (i.e., cytotoxicity) are shown in Table 1.

TABLE 1
Cytotoxicity of RPMI-8226 cells following co-incubation with
untransfected or BCMA CAR-transfected T cells

| | Untransfected | BCMA CAR | Relative increase |
|---|---|---|---|
| CD3+ | 47.4% | 54.9% | 15.8% |
| CD8+ | 48.7% | 76.2% | 56.5% |
| Relative | 2.7% | 38.8% | |
| increase | | | |

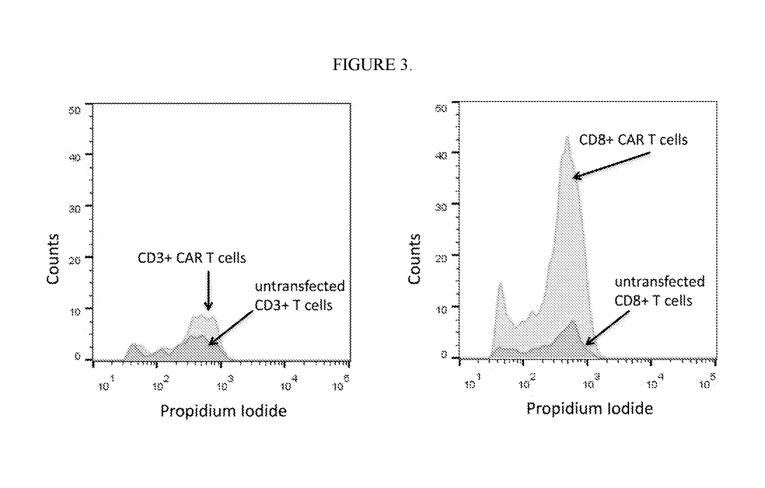

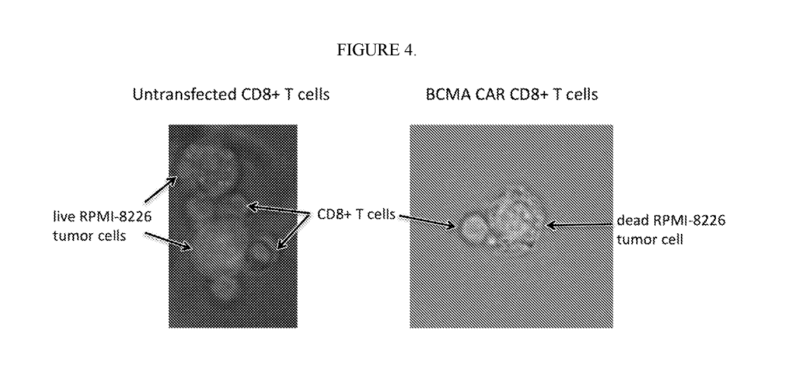

Thus, highly enriched CD8+ CAR T cells killed substantially more tumor cells than mixed CD8+/CD4+ CAR T cells (FIG. 3) and killed substantially more tumor cells than untransfected CD8+ T cells (FIG. 4). Thus, a cell therapy product comprising highly enriched CD8+ CAR T cells shows superior cytotoxic activity against multiple myeloma cells versus otherwise comparable products comprising mixed CD8+/CD4+ CAR T cells or untransfected CD8+ cells.

### Example 3

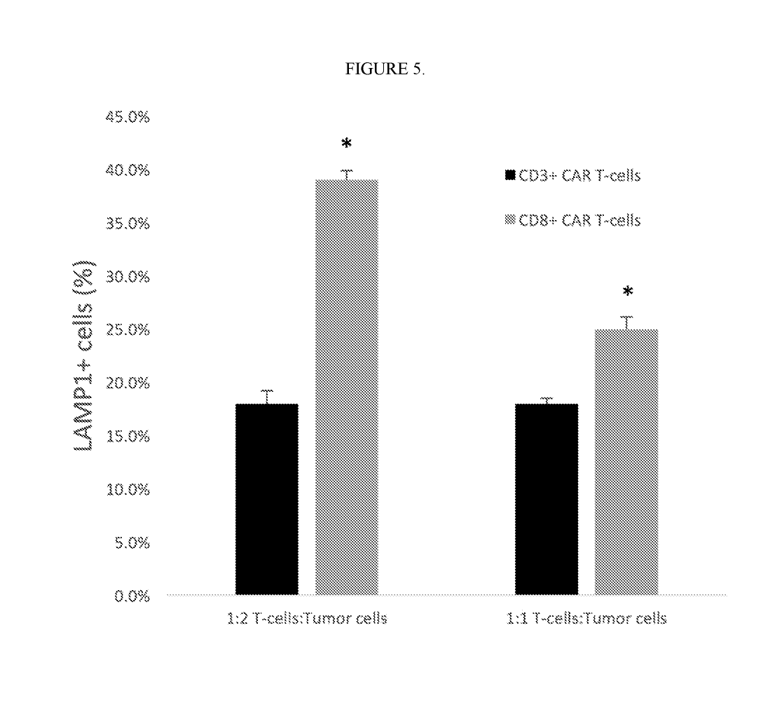

The following example shows that in response to a BCMA-expressing tumor, highly enriched CD8+ CAR T cells are more efficiently activated than mixed CD8+/CD4+ CAR T cells. Highly enriched CD8+ CAR T cells directed to BCMA were prepared according to the methods of Example 1. CD3+ CAR T cells, which comprise both CD4+ and CD8+ CAR T cells, are prepared in similar fashion. The CAR T cells are incubated in the presence of anti-LAMP1 (anti-CD107a) antibody (a marker of degranulation) with a BCMA+ myeloma cell line (MM1.S) or a BCMA− T cell line (CCRF-CEM) at 37° C. for 4 hours. CD107a immunoreactivity is assessed. CD8+ cells exhibit more degranulation CD3+ cells in response to BCMA+ myeloma cells (FIG. 5). No significant degranulation is seen in response to the negative control (BCMA−) cells. Thus, a cell therapy product comprising highly enriched CD8+ CAR T cells shows superior functional activity versus an otherwise comparable product comprising both CD4+ and CD8+ CAR T cells.

### Example 4

The following example shows that in response to a BCMA-expressing tumor, highly enriched CD8+ CAR T cells secrete less IFN-γ than mixed CD8+/CD4+ CART cells. Samples of highly enriched CD8+ CART cells and CD3+ CART cells are prepared according to the methods of Example 2. Samples are incubated overnight in the presence of a BCMA+ myeloma cell line (MM1.S) or a BCMA− T cell line (CCRF-CEM) at 37° C. Samples are then assayed for secretion of IFN-γ with a commercially available kit (Affymatrix, Inc.). Compared with the CD3+ cells, highly enriched CD8+ cells secrete substantially less IFN-γ in response to BCMA+ myeloma cells. No significant IFN-γ secretion is seen in response to the negative control (BCMA−) cells. Thus, a cell therapy product comprising highly enriched CD8+ CAR T cells shows lower IFN-γ secretion versus an otherwise comparable product comprising both CD4+ and CD8+ CAR T cells. It is expected that the lower IFN-γ secretion afforded by highly enriched CD8+ CAR T cells will correspond to better safety and tolerability in clinical use.

### Example 5

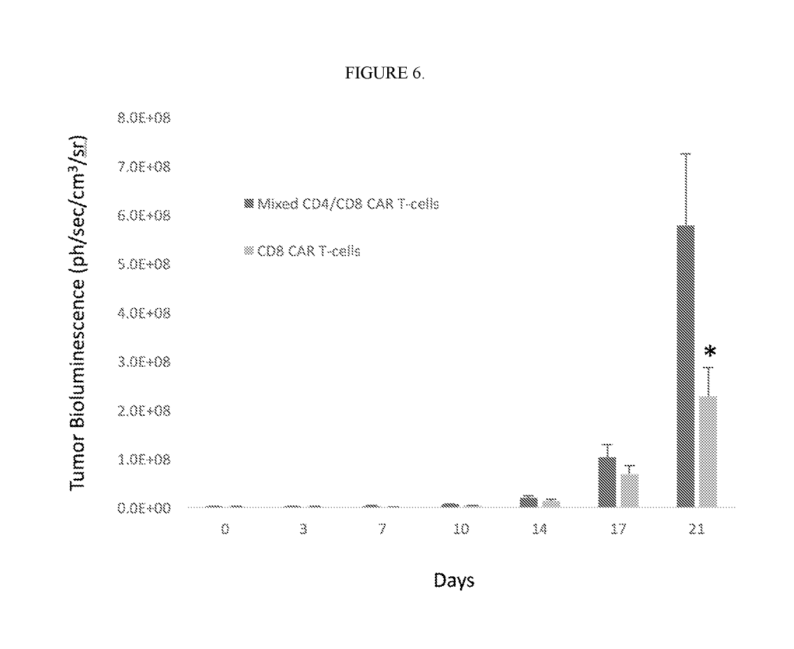

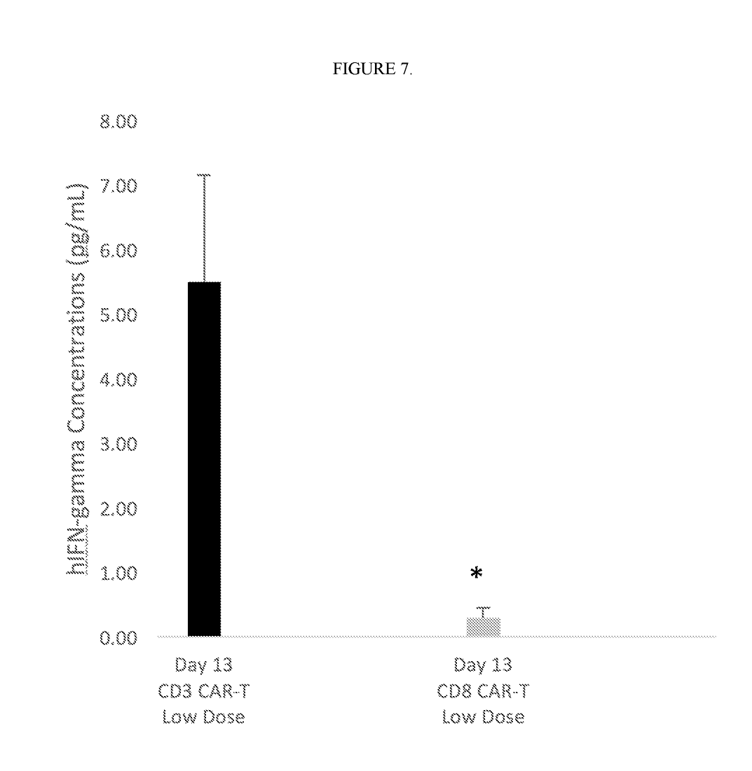

The following example demonstrates that highly enriched CD8+ CAR T cells eradicate myeloma in vivo more efficiently and while secreting less inflammatory cytokines than mixed CD8+/CD4+ CAR T cells. Highly enriched CD8+ CAR T cells directed to BCMA are prepared according to the methods of Example 1. Mixed CD3+ CAR T cells, which comprise both CD4+ and CD8+ CAR T cells, are prepared in similar fashion. The products are administered to separate groups of NSG mice injected with with luciferase-labeled MM1.S myeloma cells (MM1.S-luc cells). 8-12-week old NSG mice (NOD.Cg-Prkdc^{scid} Il2rg^{tm1Wj1}/SzJ, Jackson Labs) are randomized into 2 groups (N=7 female mice per group): intravenous BCMA CAR CD8 T-cells (group 1); or intravenous anti-BCMA CAR CD3 T-cells (group 2). Mice receive an intravenous injection of 2-5×10⁶MM1.S-luc cells in a final volume of 0.1 ml into the tail vein. Mice receive intravenous infusions of 2-5 million anti-BCMA CAR CD8 T-cells (group 1) or anti-BCMA CAR CD3 T-cells (group 2). Mice are kept for 21 days and weighed about twice per week. Tumor growth is measured with a live bioluminescence imager about twice per week Blood is collected for analysis of T-cell secreted inflammatory cytokines. Compared with mixed CD8+/CD4+ CAR T-cells, highly enriched CD8+ CAR T-cells are more effective in reducing tumor growth in vivo (FIG. 6), as measured by tumor bioluminescence. Furthermore, CD8+ CAR T cells secrete less inflammatory cytokines compared with mixed CD8+/CD4+ CAR T cells in vivo (FIG. 7). Thus, a cell therapy product comprising highly enriched CD8+ CAR T cells shows superior benefits in vivo against BCMA+ myeloma cells versus an otherwise comparable product comprising a mix of both CD4+ and CD8+ CAR T cells.

### Example 6

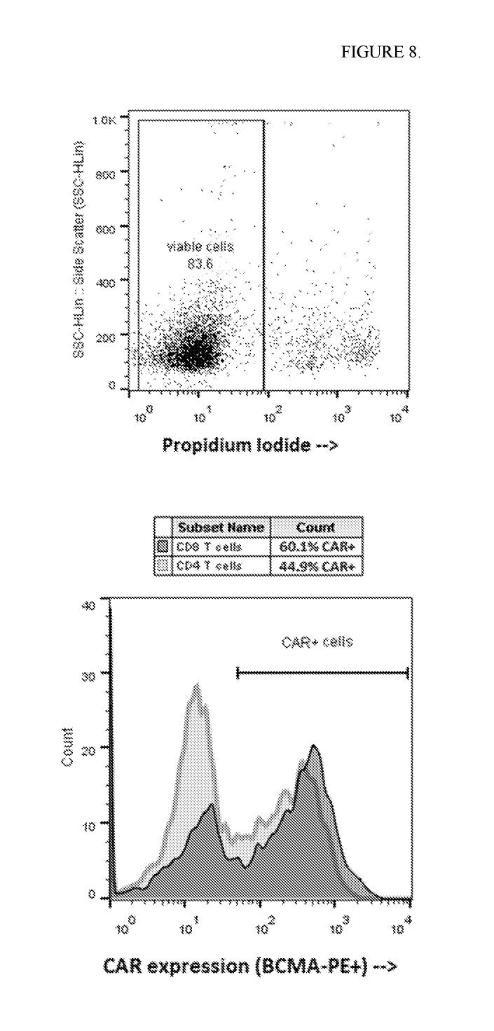

The following example shows that CD8+ T cells are more efficiently transfected with CAR mRNA compared to CD4+ T cells. CD3+ T cells that contain a mixed population of CD4+ cells and CD8+ cells were isolated, purified and transfected as described in Example 1. CD8+ cells were differentiated from CD4+ cells by flow cytometry with a monoclonal anti-CD8+ antibody. CAR expression was determined with a fluorescently-labeled protein that binds directly to the CAR (BCMA-PE). Dead cells were excluded by propidium iodide staining. CAR staining was detected with BCMA protein labeled with phycoerythrin. CD8+ cells were found to be transfected with CAR at substantially higher efficiency compared with CD4+ cells (FIG. 8). Total T cells from a healthy donor were selected using CD3 beads (Miltenyi MACS). The cells were electroporated with CAR mRNA using a Lonza Nucleofector. After a 4 hour rest to allow for CAR translation, cells were cryopreserved. The thawed cells were then measured for expression of CAR using CAR-specific antigen (BCMA-PE). Additionally cells were stained with anti-CD8-FITC (CD8 vs CD4 discrimination) and Propidium iodide (PI). The cells were then analyzed by flow cytometry (Guava EasyCyte mini). Cells were gated on viable (PI negative) and the CD8 or CD4 cells were plotted for CAR expression.

### Example 7

The following example contemplates that in response to a BCMA-expressing tumor, highly enriched CD8+ CAR T cells are more efficiently activated than mixed CD8+/CD4+ CAR T cells. Highly enriched CD8+ CAR T cells directed to BCMA are prepared according to the methods of Example 1. CD3+ CAR T cells, which comprise both CD4+ and CD8+ CAR T cells, are prepared in similar fashion. The CAR T cells are incubated in the presence of anti-CD107a antibody (a marker of degranulation) with a BCMA+ myeloma cell line (RPMI-8226) or a BCMA− T cell line (CCRF-CEM) at 37° C. for 4 hours. CD107a immunoreactivity is assessed. CD8+ cells exhibit more degranulation CD3+ cells in response to BCMA+ myeloma cells. No significant degranulation is seen in response to the negative control (BCMA−) cells. Thus, a cell therapy product comprising highly enriched CD8+ CAR T cells shows superior functional activity versus an otherwise comparable product comprising both CD4+ and CD8+ CAR T cells.

### Example 8

The following example contemplates that in response to a BCMA-expressing tumor, highly enriched CD8+ CAR T cells secrete less IFN-γ than mixed CD8+/CD4+ CART cells. Samples of highly enriched CD8+ CART cells and CD3+ CAR T cells are prepared according to the methods of Example 2. Samples are incubated overnight in the presence of a BCMA+ myeloma cell line (RPMI-8226) or a BCMA− T cell line (CCRF-CEM) at 37° C. Samples are then assayed for secretion of IFN-γ with a commercially available kit (Affymatrix, Inc.). Compared with the CD3+ cells, highly enriched CD8+ cells secrete substantially less IFN-γ in response to BCMA+ myeloma cells. No significant IFN-γ secretion is seen in response to the negative control (BCMA−) cells. Thus, a cell therapy product comprising highly enriched CD8+ CAR T cells shows lower IFN-γ secretion versus an otherwise comparable product comprising both CD4+ and CD8+ CAR T cells. It is expected that the lower IFN-γ secretion afforded by highly enriched CD8+ CAR T cells will correspond to better safety and tolerability in clinical use.

### Example 9

The following example contemplates that highly enriched CD8+ CAR T cells eradicate myeloma in vivo more efficiently than mixed CD8+/CD4+ CAR T cells. Highly enriched CD8+ CAR T cells directed to BCMA are prepared according to the methods of Example 1. CD3+ CAR T cells, which comprise both CD4+ and CD8+ CAR T cells, are prepared in similar fashion. The products are administered to separate groups of NSG mice intradermally implanted with RPMI-8226 myeloma cells. 12-week old NSG mice (NOD.Cg-Prkdc^{scid}/Il2rg^{tm1Wj1}/SzJ, Jackson Labs) are randomized into 4 groups (N=10 per group, 5 males and 5 females): intravenous BCMA CAR CD8 T-cells (group 1); intravenous anti-BCMA CAR CD3 T-cells (group 2); intravenous non-transfected CD8 T-cells (group 3); or intravenous non-transfected CD3 T-cells (group 4). Mice receive an intradermal injection of 1×10⁷ RPMI-8226 cells in a final volume of 0.1 ml into the right rear flank. About 10-14 days after injection of the RPMI-8226 cells and at least 24 hours before administration of the CAR T cells, mice receive a lymphodepleting preconditioning chemotherapy, e.g., cyclophosphamide with or without fludarabarine. Mice receive intravenous infusions of 5 million anti-BCMA CAR CD8 T-cells (group 1); anti-BCMA CAR CD3 T-cells (group 2); non-transfected CD8 T-cells (group 3); or non-transfected CD3 T-cells (group 4). Mice are kept for 14 days and weighed twice per week. Tumor area (mm²) is measured with calipers twice per week by multiplying tumor length (i.e. at the longest diameter) by width (i.e., perpendicular to length). Animals are either sacrificed at the end of the study (Day 44 from tumor implantation) or when the longest tumor length reaches 15 mm, whichever occurs earlier. Tumors are dissected from the implantation site, weighed and measured by calipers. Blood is collected for analysis of T-cell secreted inflammatory cytokines. In selected animals, live imaging is performed following implantation of luciferase-transfected RPMI-8226 cells. It is expected that compared with mixed CD8+/CD4+ CAR T cells, highly enriched CD8+ CAR T cells are more effective in eradicating tumor in vivo. Furthermore, CD8+ CAR T cells secrete less inflammatory cytokines compared with mixed CD8+/CD4+ CAR T cells in vivo. Thus, a cell therapy product comprising highly enriched CD8+ CAR T cells shows superior benefits in vivo against BCMA+ myeloma cells versus an otherwise comparable product comprising both CD4+ and CD8+ CAR T cells.

### Example 10

The following example contemplates that highly enriched CD8+ CAR T cells eradicate myeloma cells in patients with MM more efficiently and with less toxicity than mixed CD8+/CD4+ CAR T cells. Highly enriched CD8+ CAR T cells are prepared substantially according to the methods of Example 1. At least 24 hours before administration of the CAR T cells, patients receive a lymphodepleting preconditioning chemotherapy, e.g., cyclophosphamide with or without fludarabarine. Patients with MM are infused either with 1×10⁹ highly enriched CD8+ CAR T cells or with 1×10⁹ unenriched CD8+/CD4+ CAR T cells. Serum M-protein levels, free light chains of the MM-related immunoglobulin, soluble serum BCMA levels, peripheral blood CAR+ T cell counts, serum cytokine levels (e.g., IFN-γ, IL-2, IL-10), and bone marrow biopsies are analyzed at 2, 4, 8, 12 and 24 weeks after treatment. It is expected that, compared with mixed CD8+/CD4+ CAR T cells, highly enriched CD8+ CAR T cells are more effective in eradicating tumor, as measured by reduction of serum M-protein levels, free light chains of the MM-related immunoglobulin, soluble serum BCMA levels, and MM cells in bone marrow biopsies. Furthermore, it is expected that patients who receive highly enriched CD8+ T cells will experience fewer side effects than patients who receive mixed CD8+/CD4+ CAR T cells.

### Example 11

The following example contemplates that highly enriched CD8+ CAR T cells prepared by transfection of CAR mRNA eradicate myeloma cells in patients with MM with less toxicity than mixed CD8+/CD4+ CAR T cells prepared by lentiviral transduction of CAR DNA. Highly enriched CD8+ CAR T cells are prepared substantially according to the methods of Example 1. Mixed CD8+/CD4+ CAR T cells are prepared by lentiviral transduction of a corresponding CAR DNA nucleic acid construct. Patients with MM are infused either with 1×10⁹ highly enriched, RNA-transfected CD8+ CAR T cells or with 1×10⁹ unenriched, DNA-traduced CD8+/CD4+ CAR T cells. Serum M-protein levels, free light chains of the MM-related immunoglobulin, soluble serum BCMA levels, peripheral blood CAR+ T cell counts, serum cytokine levels (e.g., IFN-γ, IL-2, IL-10), and bone marrow biopsies are analyzed at 2, 4, 8, 12 and 24 weeks after treatment. It is expected that, compared with mixed, DNA-transduced CD8+/CD4+ CAR T cells, highly enriched, RNA-transfected CD8+ CAR T cells are equally or more effective in eradicating tumor, as measured by reduction of serum M-protein levels, free light chains of the MM-related immunoglobulin, soluble serum BCMA levels, and MM cells in bone marrow biopsies. Furthermore, it is expected that patients who receive highly enriched, RNA-transfected CD8+ T cells will experience fewer side effects than patients who receive mixed, DNA-transduced CD8+/CD4+ CAR T cells.

### Example 12

The following example contemplates that highly enriched CD8+ CAR T cells prepared by transfection of CAR DNA eradicate myeloma cells in patients with MM with less toxicity than mixed CD8+/CD4+ CAR T cells prepared by lentiviral transduction of CAR DNA. Highly enriched CD8+ CAR T cells are prepared substantially according to the methods of Example 1, except that a DNA nucleic acid construct corresponding to SEQ ID: 5 is used. Mixed CD8+/CD4+ CAR T cells are prepared by lentiviral transduction of a corresponding CAR DNA nucleic acid construct. At least 24 hours before administration of the CAR T cells, patients receive a lymphodepleting preconditioning chemotherapy, e.g., cyclophosphamide with or without fludarabarine. Patients with MM are infused either with 1×10⁹ highly enriched, DNA-transfected CD8+CAR T cells or with 1×10⁹ unenriched, DNA-traduced CD8+/CD4+ CAR T cells. Serum M-protein levels, free light chains of the MM-related immunoglobulin, soluble serum BCMA levels, peripheral blood CAR+ T cell counts, serum cytokine levels (e.g., IFN-γ, IL-2, IL-10), and bone marrow biopsies are analyzed at 2, 4, 8, 12 and 24 weeks after treatment. It is expected that, compared with mixed, DNA-transduced CD8+/CD4+ CAR T cells, highly enriched, DNA-transfected CD8+ CAR T cells are equally or more effective in eradicating tumor, as measured by reduction of serum M-protein levels, free light chains of the MM-related immunoglobulin, soluble serum BCMA levels, and MM cells in bone marrow biopsies. Furthermore, it is expected that patients who receive highly enriched, DNA-transfected CD8+ T cells will experience fewer side effects than patients who receive mixed, DNA-transduced CD8+/CD4+ CAR T cells.

### EQUIVALENTS AND SCOPE

Those skilled in the art will recognize, or be able to ascertain using no more than routine experimentation, many equivalents of the embodiments described herein. The scope of the present disclosure is not intended to be limited to the above description, but rather is as set forth in the appended claims.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which more than one members of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the invention encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood that, in general, where an embodiment, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Where ranges are given, any value within the range may explicitly be excluded from any one or more of the claims. Any embodiment, element, feature, application, or aspect of the compositions and/or methods of the invention, can be excluded from any one or more claims. For purposes of brevity, all of the embodiments in which one or more elements, features, purposes, or aspects is excluded are not set forth explicitly herein.

1. A cell therapy product comprising: a plurality of T cells, wherein
at least some of the T cells comprise a synthetic mRNA that encodes a chimeric antigen receptor protein;
at least 80 percent of the T cells are CD8+ cells;
at least some of the CD8+ cells express the chimeric antigen receptor protein; and
the chimeric antigen receptor protein comprises an antigen recognition moiety that binds to BCMA.

2. (canceled)

3. The product of claim 1, wherein the T cells are essentially free of CD4+ cells.

4. (canceled)

5. The product of claim 1, wherein at least 90 percent of the T cells are CD8+ cells.

6-18. (canceled)

19. The product of claim 1, wherein the antigen recognition moiety comprises the (i) heavy chain complementarity determining region (CDR)1, (ii) heavy chain CDR2, (iii) heavy chain CDR3, (iv) light chain CDR1, (v) light chain CDR2, and (vi) light chain CDR3 of any one of the amino acid sequences selected from the group consisting of SEQ ID NO: 4, SEQ ID NO: 5, SEQ ID NO: 6, SEQ ID NO: 8, SEQ ID NO: 9, SEQ ID NO: 10, SEQ ID NO: 11, and SEQ ID NO: 12.

20. The product of claim 1, wherein the antigen recognition moiety comprises the (i) heavy chain variable region and (ii) light chain variable region of any one of the amino acid sequences selected from the group consisting of SEQ ID NO: 4, SEQ ID NO: 5, SEQ ID NO: 6, SEQ ID NO: 8, SEQ ID NO: 9, SEQ ID NO: 10, SEQ ID NO: 11, and SEQ ID NO: 12.

21. The product of claim 1, wherein the product further comprises a pharmaceutically acceptable carrier.

22. A method for producing a cell therapy product, the method comprising:
(i) purifying CD8+ T cells; and
(ii) transfecting the CD8+ T cells with a synthetic RNA construct encoding a chimeric antigen receptor (CAR) protein, wherein the CAR protein comprises an antigen recognition moiety that binds to BCMA.

23. The method of claim 22, wherein the step of purifying comprises negative selection.

24. The method of claim 22, wherein the step of purifying comprises positive selection.

25. The method of claim 22, wherein the step of transfecting comprises electroporation.

26. The method of claim 22, wherein the CD8+ T cells purified in step (i) are essentially free of CD4+ cells.

27. The method of claim 22, wherein at least 90 percent of T cells purified in step (i) are CD8+ cells.

28. The method of claim 22, wherein the antigen recognition moiety comprises the (i) heavy chain complementarity determining region (CDR)1, (ii) heavy chain CDR2, (iii) heavy chain CDR3, (iv) light chain CDR1, (v) light chain CDR2, and (vi) light chain CDR3 of any one of the amino acid sequences selected from the group consisting of SEQ ID NO: 4, SEQ ID NO: 5, SEQ ID NO: 6, SEQ ID NO: 8, SEQ ID NO: 9, SEQ ID NO: 10, SEQ ID NO: 11, and SEQ ID NO: 12.

29. The method of claim 22, wherein the antigen recognition moiety comprises the (i) heavy chain variable region and (ii) light chain variable region of any one of the amino acid sequences selected from the group consisting of SEQ ID NO: 4, SEQ ID NO: 5, SEQ ID NO: 6, SEQ ID NO: 8, SEQ ID NO: 9, SEQ ID NO: 10, SEQ ID NO: 11, and SEQ ID NO: 12.

30. The method of claim 22 further comprising formulating the cells of step (ii) into a pharmaceutical composition comprising a pharmaceutically acceptable carrier.

31. A method for treating a B cell malignancy in an individual, the method comprising administering to the individual the product of claim 1.

32. A method for treating myeloma in an individual, the method comprising administering to the individual a product comprising a plurality of T cells, wherein:
at least 10% of the T cells comprise a synthetic mRNA that encodes a chimeric antigen receptor protein;
at least 80 percent of the T cells are CD8+ cells;
at least some of the CD8+ cells express the chimeric antigen receptor protein; and
the chimeric antigen receptor protein comprises an antigen recognition moiety that binds to BCMA.

33. The method of claim 32, wherein the T cells are essentially free of CD4+ cells.

34. The method of claim 32, wherein at least 90 percent of the T cells are CD8+ cells.

35. The method of claim 32, wherein the product is prepared by a process comprising the steps of purifying CD8+ T cells and transfecting the CD8+ T cells with a synthetic RNA construct encoding the chimeric antigen receptor protein.
